(19) United States
(12) Patent Application Publication
(10) Pub. No.: US 2026/0345463 A1
(43) Pub. Date: Nov. 5, 2026

(54) COMPOSITIONS COMPRISING WATER, CATIONIC ALPHA-1,6-GLUCAN ETHER, AND ORGANIC SOLVENT

(71) Applicant: Nutrition & Biosciences USA 4, Inc., Wilmington, DE (US)

(72) Inventors: Michael D. Gagnon, West Grove, PA (US); Helen S. M. Lu, Wallingford, PA (US); Ilkka Ilari Kruus, Oegstgeest (NL); Takenori Kashimura, Tokyo (JP); Mark Robert Sivik, Mason, OH (US); Wouter Walravens, Humbeek (BE); Karel Jozef Maria Depoot, Anzegem (BE); Antonio Quintieri, Waterloo (BE); Kristine Lynn Niehaus, Harrison, OH (US); Katrien Van Roey, Werchter (BE)

(21) Appl. No.: 19/117,709

(22) PCT Filed: Oct. 12, 2023
(86) PCT No.: PCT/US2023/076672

§ 371 Date: Apr. 2, 2025

Related U.S. Application Data
(60) Provisional application No. 63/379,507, filed on Oct. 14, 2022.
(60) Provisional application No. 63/587,488, filed on Oct. 3, 2023.

(51) Int. Cl. A61K 8/73; A61K 8/20; A61K 8/34; A61K 8/362; A61K 8/44; A61K 8/46; A61K 8/81; A61Q 5/02; A61Q 19/10; C11D 1/58

(52) U.S. Cl. CPC A61K 8/732; A61K 8/20; A61K 8/34; A61K 8/362; A61K 8/44; A61K 8/442; A61K 8/463; A61K 8/731; A61K 8/737; A61K 8/817; A61Q 5/02; A61Q 19/10; C11D 1/58

(57) ABSTRACT

Compositions are disclosed herein comprising (i) about 20-70 wt % of at least one organic solvent, (ii) about 20-50 wt % of at least one cationic alpha-glucan ether derivative, and (iii) less than about 45 wt % water. At least about 50% of the glycosidic linkages of the cationic alpha-glucan ether derivative are alpha-1,6 linkages and the ether derivative has a degree of substitution (DoS) of about 0.001 to about 3.0 with at least one ether-linked positively charged organic group. Further disclosed are methods for producing these compositions, as well as using them as ingredients in various products.

This application claims the benefit of U.S. Provisional Appl. Nos. 63/379,507 (filed Oct. 14, 2022) and 63/587,488 (filed Oct. 3, 2023), which are each incorporated herein by reference in their entirety.

### FIELD

The present disclosure is in the field of polysaccharide derivatives. For example, the disclosure pertains to aqueous compositions comprising one or more cationic alpha-glucan ether derivatives such as a cationic alpha-1,6-glucan ether derivative, and use thereof in various applications.

### BACKGROUND

Driven by a desire to find new structural polysaccharides using enzymatic syntheses or genetic engineering of microorganisms, researchers have discovered oligosaccharides and polysaccharides that are biodegradable and that can be made economically from renewably-sourced feedstocks. Further work has shown that such polysaccharides can be chemically modified (derivatized) to have additional utilities in areas such as personal care, household care, industrial care, pharmaceuticals and food. For example, ethers and esters of alpha-glucan comprising alpha-1,3 glycosidic linkages have been disclosed to have various applications (e.g., U.S. Patent Appl. Publ. Nos. 2016/0304629, 2016/0311935, 2017/0204232, 2014/0187767, 2020/0308371). Various derivatives of alpha-glucan comprising alpha-1,6 glycosidic linkages, and applications for use thereof, have also been disclosed (e.g., U.S. Patent Appl. Publ. Nos. 2018/0312781, 2018/0237816, 2018/0282385).

Cationic alpha-1,6-glucan ethers exhibit various benefical effects such as surface deposition and modification. Despite this utility, aqueous compositions comprising cationic alpha-1,6-glucan ethers can sometimes be challenging to process and handle. Modified aqueous compositions comprising one or more cationic alpha-1,6-glucan ethers are disclosed herein that address this issue.

### SUMMARY

In one embodiment, the present disclosure concerns a composition comprising:
(i) about 15% to 75% by weight of at least one organic solvent,
(ii) about 20% to 50% by weight of at least one cationic alpha-glucan ether derivative, and
(iii) less than about 50% by weight water,
wherein at least about 50% of the glycosidic linkages of the cationic alpha-glucan ether derivative are alpha-1,6 linkages, and the cationic alpha-glucan ether derivative has a degree of substitution (DoS) of about 0.001 to about 3.0 with at least one positively charged organic group that is ether-linked to the alpha-glucan.

In another embodiment, the present disclosure concerns a product comprising a composition as presently disclosed, typically wherein the composition was used as an ingredient or component in producing the product.

In another embodiment, the present disclosure concerns method/process of producing a composition as presently disclosed. Such a method/process can comprise: (a) providing an aqueous composition comprising a cationic alpha-glucan ether derivative herein, (b) mixing, into the aqueous composition, an organic solvent herein, and (c) optionally concentrating the cationic alpha-glucan ether derivative and the organic solvent in the aqueous composition following step (b).

### BRIEF DESCRIPTION OF THE DRAWINGS

FIG. 1: Various cationic etherification reaction impurities that can be in a liquid composition in some aspects are presented.

FIG. 2: Various possible impurities that can be in a liquid composition in some aspects are presented.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like. The numerical values of the various ranges in the present disclosure, unless expressly indicated otherwise, are stated as approximations as though the minimum and maximum values within the stated ranges were both proceeded by the word “about”. In this manner, slight variations above and below the stated ranges can typically be used to achieve substantially the same results as values within the ranges. Also, the disclosure of these ranges is intended as a continuous range including each and every value between the minimum and maximum values.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

It is to be appreciated that certain features of the present disclosure, which are, for clarity, described above and below in the context of aspects/embodiments, may also be provided in combination in a single element. Conversely, various features of the disclosure that are, for brevity, described in the context of a single aspect/embodiment, can also be provided separately or in any sub-combination.

The term “polysaccharide” (or “glycan”) means a polymeric carbohydrate molecule composed of long chains of monosaccharide units bound together by glycosidic linkages and on hydrolysis gives the polysaccharide's constituent monosaccharides and/or oligosaccharides. A polysaccharide herein can be linear or branched, and/or can be a homopolysaccharide (comprised of only one type of constituent monosaccharide) or heteropolysaccharide (comprised of two or more different constituent monosaccharides). Examples of polysaccharides herein include glucan (polyglucose) and soy polysaccharide.

A “glucan” herein is a type of polysaccharide that is a polymer of glucose (polyglucose). A glucan can be comprised of, for example, about, or at least about, 90%, 91%, 92%, 93%, 94%, 95%, 96%, 97%, 98%, 99%, or 100% by weight glucose monomeric units. Examples of glucans herein are alpha-glucan and beta-glucan.

The terms “alpha-glucan”, “alpha-glucan polymer” and the like are used interchangeably herein. An alpha-glucan is a polymer comprising glucose monomeric units linked together by alpha-glycosidic linkages. In typical aspects, the glycosidic linkages of an alpha-glucan herein are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. An example of an alpha-glucan polymer herein is alpha-1,6-glucan.

The terms “alpha-1,6-glucan”, “poly alpha-1,6-glucan”, “alpha-1,6-glucan polymer”, “dextran”, and the like herein refer to a water-soluble alpha-glucan comprising glucose monomeric units linked together by glycosidic linkages, wherein at least about 50% of the glycosidic linkages are alpha-1,6. Alpha-1,6-glucan in some aspects comprises about, or at least about, 90%, 95%, or 100% alpha-1,6 glycosidic linkages. Other linkages that can be present in alpha-1,6-glucan include alpha-1,2, alpha-1,3, and/or alpha-1,4 linkages.

An “alpha-1,2 branch” (and like terms) as referred to herein typically comprises a glucose that is alpha-1,2-linked to a dextran backbone; thus, an alpha-1,2 branch herein can also be referred to as an alpha-1,2,6 linkage. An alpha-1,2 branch herein typically has one glucose group (can optionally be referred to as a pendant glucose).

An “alpha-1,3 branch” (and like terms) as referred to herein typically comprises a glucose that is alpha-1,3-linked to a dextran backbone; thus, an alpha-1,3 branch herein can also be referred to as an alpha-1,3,6 linkage. An alpha-1,3 branch herein typically has one glucose group (can optionally be referred to as a pendant glucose).

An “alpha-1,4 branch” (and like terms) as referred to herein typically comprises a glucose that is alpha-1,4-linked to a dextran backbone; thus, an alpha-1,4 branch herein can also be referred to as an alpha-1,4,6 linkage. An alpha-1,4 branch herein typically has one glucose group (can optionally be referred to as a pendant glucose).

The percent branching in an alpha-glucan herein typically refers to that percentage of all the linkages in the alpha-glucan that represent branch points. For example, the percent of alpha-1,2 branching in an alpha-glucan herein refers to that percentage of all the linkages in the glucan that represent alpha-1,2 branch points. Except as otherwise noted, linkage percentages disclosed herein are based on the total linkages of an alpha-glucan, or the portion of an alpha-glucan for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages).

The glycosidic linkage profile of an alpha-glucan or derivative thereof can be determined using any method known in the art. For example, a linkage profile can be determined using methods using nuclear magnetic resonance (NMR) spectroscopy (e.g., ¹³C NMR and/or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The term “molar substitution” (M.S.) as used herein refers to the moles of an organic group per monomeric unit of an alpha-glucan derivative herein. It is noted that the molar substitution value for an alpha-glucan derivative, for example, may have a very high upper limit, for example in the hundreds or even thousands.

The “molecular weight” of an alpha-glucan or alpha-glucan derivative herein can be represented as weight-average molecular weight (Mw) or number-average molecular weight (Mn), the units of which are in Daltons (Da) or grams/mole. Alternatively, molecular weight can be represented as DPw (weight average degree of polymerization) or DPn (number average degree of polymerization). The molecular weight of smaller alpha-glucan polymers such as oligosaccharides can optionally be provided as “DP” (degree of polymerization), which simply refers to the number of monomers comprised within the alpha-glucan; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi; where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=ΣNiMi/ΣNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPn and DPw can be calculated from Mn and Mw, respectively, by dividing them by molar mass of the one monomer unit M₁. In the case of unsubstituted glucan polymer, M₁=162. In the case of a substituted (derivatized) glucan polymer, M₁=162+M_{f}×DoS, where M_{f} is molar mass of the substituting group, and DoS is degree of substitution (average number of substituted groups per one glucose unit of the glucan polymer).

An “alpha-glucan derivative” (and like terms) herein typically refers to an alpha-glucan that has been substituted with at least one type of organic group. The degree of substitution (DoS) of an alpha-glucan derivative can be up to about 3.0 (e.g., about 0.001 to about 3.0) in some aspects. An organic group herein that is an ether group is linked to an alpha-glucan derivative via ether linkage. A precursor of an alpha-glucan derivative herein typically refers to the non-derivatized alpha-glucan used to make the derivative (can also be referred to as the alpha-glucan or alpha-glucan portion of the derivative). An organic group herein typically is positively charged (cationic); generally, such charge can be as it exists when the organic group is in an aqueous composition herein, further taking into account the pH of the aqueous composition (in some aspects, the pH can be 4-10, 5-9, 6-8, or any pH as disclosed herein).

The term “degree of substitution” (DoS, or DS) as used herein refers to the average number of hydroxyl groups that are substituted with one or more types of organic group (e.g., via an ether linkage) in each monomeric unit of an alpha-glucan derivative. The DoS of an alpha-glucan derivative herein can be stated with reference to the DoS of a specific substituent, or the overall DoS, which is the sum of the DoS values of different substituent types (e.g., if a mixed ether). Unless otherwise disclosed, when DoS is not stated with reference to a specific substituent type, the overall DoS is meant.

Terms used herein regarding “ethers” (e.g., alpha-glucan ether derivative) can be as disclosed, for example, in U.S. Patent Appl. Publ. Nos. 2016/0311935, 2018/0237816, or 2020/0002646, or International Pat. Appl. Publ. No. WO2021/257786, which are each incorporated herein by reference. The terms “alpha-glucan ether derivative”, “alpha-glucan ether compound”, “alpha-glucan ether”, and the like are used interchangeably herein. An alpha-glucan ether derivative herein is alpha-glucan that has been etherified with one or more organic groups (e.g., charged organic group such as cationic group) such that the derivative has a DoS with one or more organic groups of up to about 3.0. An alpha-glucan ether derivative is termed an “ether” herein by virtue of comprising the substructure —C_{G}—O—C—, where “—C_{G}—” represents a carbon atom of a monomeric unit (typically glucose) of the alpha-glucan ether derivative (where such carbon atom was bonded to a hydroxyl group [—OH] in the alpha-glucan precursor of the ether), and where “—C—” is a carbon atom of an organic group.

An organic group can refer to a “positively charged organic group”. A positively charged organic group as used herein refers to one or more carbons (e.g., “carbon chain”) that has one or more hydrogens substituted with another atom or functional group (i.e., a “substituted alkyl group”), where one or more of the substitutions is with a positively charged group. Where a positively charged organic group has a substitution in addition to a substitution with a positively charged group, such additional substitution may be with one or more hydroxyl groups, oxygen atoms (thereby forming an aldehyde or ketone group), alkyl groups, and/or additional positively charged groups. A positively charged organic group has a net positive charge since it comprises one or more positively charged groups. The terms “positively charged group”, “positively charged ionic group”, “cationic group” and the like are used interchangeably herein. A positively charged group comprises a cation (a positively charged ion). Examples of positively charged groups include substituted ammonium groups, carbocation groups and acyl cation groups.

The terms “substituted ammonium”, “substituted ammonium group”, “substituted ammonium ion”, “substituted ammonium cation” and the like are used interchangeably herein. A substituted ammonium group herein comprises Structure I:

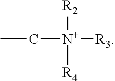

R₂, R₃ and R₄ in Structure I each independently represent a hydrogen atom or an alkyl, aryl, cycloalkyl, aralkyl, or alkaryl group. The positioning of R₂, R₃ and R₄ in Structure I is generally of no particular importance and not intended to invoke any particular stereochemistry. The carbon atom (C) in Structure I is part of one or more carbons (e.g., “carbon chain”) of the positively charged organic group. The carbon atom is either directly ether-linked to a glucose monomeric unit of an alpha-glucan herein, or is part of a chain of two or more carbon atoms that is ether-linked to the glucose monomeric unit. The carbon atom in Structure I can be —CH₂—, —CH— (where an H is substituted with another group such as a hydroxy group), or —C— (where both H's are substituted).

A substituted ammonium group herein can be a “primary ammonium group”, “secondary ammonium group”, “tertiary ammonium group”, or “quaternary ammonium” group, depending on the composition of R₂, R₃ and R₄ in Structure I. A primary ammonium group herein refers to Structure I in which each of R₂, R₃ and R₄ is a hydrogen atom (i.e., —C—NH₃⁺). A secondary ammonium group herein refers to Structure I in which each of R₂ and R₃ is a hydrogen atom and R₄ is an alkyl, aryl, cycloalkyl, aralkyl, or alkaryl group. A tertiary ammonium group herein refers to Structure I in which R₂ is a hydrogen atom and each of R₃ and R₄ is an alkyl, aryl, cycloalkyl, aralkyl, or alkaryl group. Assignment herein of R₂, R₃ and R₄ is completely arbitrary. A quaternary ammonium group herein refers to Structure I in which each of R₂, R₃ and R₄ is independently an alkyl, aryl, cycloalkyl, aralkyl, or alkaryl group (i.e., none of R₂, R₃ and R₄ is a hydrogen atom). It would be understood that a fourth member (i.e., R₁) implied by the above nomenclature is the one or more carbons (e.g., chain) of the positively charged organic group that is ether-linked to a glucose monomeric unit of the alpha-glucan.

Examples of substituted ammonium alpha-glucan ethers herein comprise a hydroxypropyl group that links the ammonium group to the alpha-glucan. The positively charged organic group of such an ether compound can be represented as Structure II:

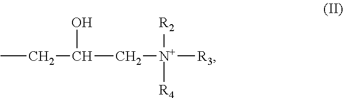

where each of R₂, R₃ and R₄ is as described above for either a primary, secondary, tertiary, or quaternary ammonium group.

The terms “etherification reaction”, “etherification reaction composition” and the like herein refer to a reaction comprising water, at least one alpha-glucan as presently disclosed, and an etherification agent. These components are typically dissolved and/or mixed under alkaline conditions (typically, in an aqueous solvent comprising alkali hydroxide). A reaction is placed under suitable conditions (e.g., time, temperature, pH) for the etherification agent to etherify one or more hydroxyl groups of glucose monomeric units of alpha-glucan with an organic group herein, thereby yielding an alpha-glucan ether compound. A reaction that has commenced and contains at least some amount of an alpha-glucan ether product can likewise be referred to as an etherification reaction, or as the case may be, a completed etherification reaction.

The term “alkaline conditions” herein, such as for an etherification reaction composition, refers to a solution or mixture pH of at least 11 or 12. Alkaline conditions can be prepared by any means known in the art, such as by dissolving an alkali hydroxide in an aqueous composition.

The terms “etherification agent”, “alkylation agent” and the like are used interchangeably herein. An etherification agent herein refers to an agent that can be used to etherify one or more hydroxyl groups of one or more glucose monomeric units of an alpha-glucan with an organic group. An etherification agent thus comprises at least one organic group.

An alpha-glucan or ether derivative thereof that is “aqueous-soluble” or “water-soluble” (and like terms) herein dissolves (or appreciably dissolves) in water or other aqueous conditions, optionally where the aqueous conditions are further characterized to have a pH of 4-9 (e.g., pH 6-8) and/or temperature of about 1 to 130 °C (e.g., 20-25 °C). In some aspects, an aqueous-soluble alpha-glucan or ether derivative thereof is soluble at 1% by weight or higher in pH 7 water at 25 °C. In contrast, an alpha-glucan or ether derivative thereof that is “aqueous-insoluble” or “water-insoluble” (and like terms) does not dissolve under these conditions. In some aspects, less than 1.0 gram (e.g., no detectable amount) of an aqueous-insoluble alpha-glucan or ether derivative thereof dissolves in 1000 milliliters of such aqueous conditions (e.g., water at 23 °C). Alpha-glucan and alpha-glucan ether derivatives of the present disclosure typically are aqueous-soluble.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa-s), for example. A centipoise is one one-hundredth of a poise; one poise is equal to 0.100 kg·m⁻¹·s⁻¹. The terms “viscosity modifier”, “viscosity-modifying agent” and the like herein refer to anything that can alter/modify the viscosity of a fluid or aqueous composition.

The terms “polar organic solvent” and “water-miscible organic solvent” (and like terms) are used interchangeably herein. A polar organic solvent can be dissolved in water or an aqueous solution. Thus, a polar organic solvent does not separate out into a different phase when added to water or an aqueous solution. A polar organic solvent contains carbon and at least one heteroatom (i.e., non-carbon or -hydrogen atom) such as oxygen, nitrogen, sulfur, or phosphorous. This contrasts with non-polar organic solvents, which generally comprise only carbon and hydrogen atoms. A polar organic solvent typically has a dielectric constant greater than about 4. A polar organic solvent contains dipoles due to polar bonds.

The term “protic polar organic solvent” (and like terms) herein refers to a polar organic solvent that has one or more suitably labile hydrogen atoms that can form hydrogen bonds. A protic polar organic solvent generally contains hydrogen atoms bonded to an atom with electronegative character, e.g., there are one or more O—H, N—H, and/or S—H bonds.

The terms “fiber”, “fibers” and the like herein can refer to staple fibers (staple length fibers) or continuous fibers, in some aspects. Fibers herein can comprise alpha-1,3-glucan, natural fiber (e.g., cellulose, cotton, wool, silk), or synthetic fiber (e.g., polyester), or any other type of material disclosed herein that can form a fiber. Fibers can be in a fiber-containing material/article/composition, for example, such as a woven or non-woven product.

The term “woven product” and like terms herein refer to a product formed by weaving, braiding, interlacing, or otherwise intertwining threads or fibers in an organized, consistent, and/or repeating manner.

The terms “non-woven”, “non-woven product”, “non-woven web” and the like herein refer to a web of individual fibers or filaments that are interlaid, typically in a random or unidentifiable manner. This contrasts with a knitted or woven fabric, which has an identifiable network of fibers or filaments. In some aspects, a non-woven product comprises a non-woven web that is bound or attached to another material such as a substrate or backing.

The terms “fabric”, “textile”, “cloth” and the like are used interchangeably herein to refer to a woven material having a network of natural and/or artificial fibers. Such fibers can be in the form of thread or yarn, for example.

The term “household care product” and like terms typically refer to products, goods and services relating to the treatment, cleaning, caring and/or conditioning of a home and its contents. The foregoing include, for example, chemicals, compositions, products, or combinations thereof having application in such care.

A “fabric care composition” and like terms refer to any composition suitable for treating fabric in some manner. Examples of such a composition include laundry detergents and fabric softeners, which are examples of laundry care compositions.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The terms “heavy duty detergent”, “all-purpose detergent” and the like are used interchangeably herein to refer to a detergent useful for regular washing of white and/or colored textiles at any temperature. The terms “low duty detergent”, “fine fabric detergent” and the like are used interchangeably herein to refer to a detergent useful for the care of delicate fabrics such as viscose, wool, silk, microfiber, or other fabric requiring special care. “Special care” can include conditions of using excess water, low agitation, and/or no bleach, for example.

The terms “fabric softener”, “fabric conditioner” and the like herein refer to compositions, such as in liquid or solid form, that deposit lubricants and/or other surface-modifying ingredients onto fabric to, for example, help maintain softness of the fabric and/or provide other beneficial features to fabric (e.g., lubricity, anti-static, anti-cling, and/or anti-wrinkling). A fabric softener herein typically is applied to fabric following fabric washing with a laundry detergent, usually while rinsing the fabric.

The term “personal care product” and like terms typically refer to products, goods and services relating to the treatment, cleaning, cleansing, caring or conditioning of a person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

The term “medical product” and like terms typically refer to products, goods and services relating to the diagnosis, treatment, and/or care of patients.

The terms “film”, “sheet” and like terms herein refer to a generally thin, continuous material. A film can be comprised as a layer or coating on a material, or can be alone (e.g., not attached to a material surface, free-standing). A “coating” (and like terms) as used herein refers to a layer covering a surface of a material. The term “uniform thickness” as used to characterize a film or coating herein can refer to a contiguous area that (i) is at least 20% of the total film/coating area, and (ii) has a standard deviation of thickness of less than about 50 nm, for example. The term “continuous layer” means a layer of a composition applied to at least a portion of a substrate, wherein a dried layer of the composition covers ≥99% of the surface to which it has been applied and having less than 1% voids in the layer that expose the substrate surface. The ≥99% of the surface to which the layer has been applied excludes any area of the substrate to which the layer has not been applied. A coating herein can make a continuous layer in some aspects. A coating composition (and like terms) herein refers to all the solid components that form a layer on a substrate, such as an alpha-glucan ether derivative herein and, optionally, pigment, surfactant, dispersing agent, binder, crosslinking agent, and/or other additives.

The term “industrial product” and like terms typically refer to products, goods and services used in industrial and/or institutional settings, but typically not by individual consumers.

The terms “sequence identity”, “identity” and the like as used herein with respect to a polypeptide amino acid sequence (e.g., that of a glucosyltransferase) are as defined and determined in U.S. Patent Appl. Publ. No. 2017/0002336, which is incorporated herein by reference.

A composition herein that is “dry” or “dried” typically has less than 1 wt % water comprised therein.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The term “isolated” means a substance (or process) in a form or environment that does not occur in nature. A non-limiting example of an isolated substance includes any alpha-glucan ether derivative disclosed herein. It is believed that the embodiments disclosed herein are synthetic/man-made (could not have been made or practiced except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some aspects of the present disclosure concern a composition that comprises:
(i) about 15% to 75% by weight of at least one organic solvent,
(ii) about 20% to 50% by weight of at least one cationic alpha-glucan ether derivative (i.e., an ether derivative of an alpha-glucan herein), and
(iii) less than about 50% by weight water (but typically at least about 20% by weight water);
   wherein at least about 50% of the glycosidic linkages of the cationic alpha-glucan ether derivative are alpha-1,6 linkages, and the cationic alpha-glucan ether derivative has a degree of substitution (DoS) of about 0.001 to about 3.0 with at least one positively charged organic group that is ether-linked to the alpha-glucan. Such a composition can allow for more facile processing and handling of a cationic alpha-glucan ether derivative of the disclosure in aqueous formats. In some aspects, such a composition can be used as an ingredient in preparing a product comprising a cationic alpha-glucan ether derivative. A composition of the disclosure can optionally be characterized as a liquid composition.

In some aspects, a liquid composition of the present disclosure comprises:
(i) about 15%, 20%, 25%, 30%, 31%, 32%, 33%, 34%, 35%, 36%, 37%, 38%, 39%, 40%, 45%, 50%, 55%, 60%, 65%, 70%, 75%, 15-75%, 15-70%, 15-65%, 15-60%, 15-55%, 15-50%, 15-45%, 15-40%, 15-35%, 15-30%, 15-25%, 20-75%, 20-70%, 20-65%, 20-60%, 20-55%, 20-50%, 20-45%, 20-40%, 20-35%, 20-30%, 20-25%, 25-75%, 25-70%, 25%-65%, 25-60%, 25-55%, 25-50%, 25-45%, 25-40%, 25-35%, 25-30%, 30-75%, 30-70%, 30%-65%, 30-60%, 30-55%, 30-50%, 30-45%, 30-40%, 30-35%, or 32-35% by weight of at least one organic solvent herein,
(ii) about 20%, 25%, 30%, 31%, 32%, 33%, 34%, 35%, 36%, 37%, 38%, 39%, 40%, 45%, 50%, 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, or 32-35% by weight of at least one cationic alpha-glucan ether derivative herein, and
(iii) about, or less than about, 50%, 45%, 40%, 39%, 38%, 37%, 36%, 35%, 34%, 33%, 32%, 31%, 30%, 25%, 20%, 20-50%, 20-45%, 20-40%, 20-35%, 20-30%, 20-25%, 25-50%, 25-45%, 25-40%, 25-35%, 25-30%, 30-45%, 30-40%, 30-35%, or 32-35% by weight water.

Any combination of wt % values/ranges taken from (i), (ii) and (iii) above can characterize a liquid composition herein. Merely as examples, a liquid composition herein can comprise:
(A) (i) 25-40 wt % organic solvent(s), (ii) 25-40 wt % cationic alpha-glucan ether(s) and (iii) 25-40 wt % water;
(B) (i) 25-35 wt % organic solvent(s), (ii) 25-35 wt % cationic alpha-glucan ether(s) and (iii) 25-35 wt % water;
(C) (i) 30-40 wt % organic solvent(s), (ii) 30-40 wt % cationic alpha-glucan ether(s) and (iii) 30-40 wt % water;
(D) (i) 30-35 wt % organic solvent(s), (ii) 30-35 wt % cationic alpha-glucan ether(s) and (iii) 30-35 wt % water;
(E) (i) 32-35 wt % organic solvent(s), (ii) 32-35 wt % cationic alpha-glucan ether(s) and (iii) 32-35 wt % water;
(F) (i) 32-34 wt % organic solvent(s), (ii) 32-34 wt % cationic alpha-glucan ether(s) and (iii) 32-34 wt % water; or
(G) (i) 33-34 wt % organic solvent(s), (ii) 33-34 wt % cationic alpha-glucan ether(s) and (iii) 33-34 wt % water.

In some aspects, the combination of (i), (ii) and (iii) constitutes about, or at least about, 80%, 85%, 90%, 95%, 96%, 97%, 98%, 99%, 99.5%, or 100% by weight of the composition. Thus, in some aspects, one or more additional components can be present in a liquid composition comprising (i), (ii) and (iii). Examples of other components can be any as disclosed herein, such as one or more of a salt, buffer, enzyme, sugar and/or other saccharide, impurity, byproduct, and/or preservative.

One, two, three or more organic solvents can make up the organic solvent constituent of a liquid composition, for example. Typically, one or more organic solvents herein are polar organic solvents, which are soluble/miscible in water; a polar organic solvent(s) can be protic, for example. Examples of suitable organic solvents herein include ethanol, ethylene glycol, polyethylene glycol, 1,2-propanediol, propylene glycol, dipropylene glycol, tripropyleneglycol, polypropylene glycol, and/or glycerol.

A liquid composition of the disclosure comprising (i), (ii) and (iii) above typically exists in a completely liquid state (i.e., there are no solids present; the cationic alpha-glucan ether[s] is completely dissolved). This liquid state typically exists in a single phase, as suitable organic solvents herein are miscible in water under conditions disclosed herein.

A liquid composition of the disclosure comprising (i), (ii) and (iii) in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, glucose, leucrose, and/or soluble gluco-oligosaccharides, for example. A liquid composition in some aspects can have one or more salts/buffers (e.g., Na⁺, Cl⁻, NaCl, phosphate, tris, citrate) (e.g., ≤0.1, 0.5, 1.0, 2.0, or 3.0 wt %), and/or a pH of about 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 5.0-10.0, 5.0-9.0, 5.0-8.0, 6.0-10.0, 6.0-9.0, or 6.0-8.0, for example.

The temperature of a liquid composition herein comprising (i), (ii) and (iii) herein can be about, at least about, or less than about, 0, 5, 10, 15, 20, 25, 30, 35, 37, 40, 42, 45, 50, 55, 60, 65, 70, 75, 80, 85, 90, 95, 100, 105, 110, 115, 120, 125, 130, 10-30, 10-25, 15-50, 15-30, 15-25, 20-40, 20-35, 20-30, 20-25, 25-30, 30-50, 30-45, 30-40, 30-35, 35-40, 35-50, 40-45, 50-60, 110-130, 110-125, 110-120, 115-130, or 115-125 °C, for example.

Liquid composition component (ii) can comprise one, two, three, four or more different cationic alpha-glucan ether derivatives herein, for example. A non-derivatized alpha-glucan (e.g., precursor compound[s] of the cationic alpha-glucan ether derivative[s]) typically is not present and/or not detectable (e.g., is not detectable down to a level of about, or less than about, 0.01, 0.005, 0.001, or 0.0005 wt %), for example, in a liquid composition.

In some aspects, a cationic alpha-glucan ether comprises about, or at least about, 50%, 60%, 70%, 80%, 85%, 90%, 91%, 92%, 93%, 94%, 95%, 96%, 97%, 98%, 99%, 99.5%, or 100% alpha-1,6 glycosidic linkages (i.e., the ether is a cationic alpha-1,6-glucan ether, or cationic dextran ether). In some aspects, a substantially linear dextran ether can comprise 5%, 4%, 3%, 2%, 1%, 0.5% or less glycosidic branches (a linear dextran ether has 100% alpha-1,6 linkages). If present, glycosidic branches from a dextran ether are typically short, being one (pendant), two, or three glucose monomers in length. In some aspects, a dextran ether can comprise about, or less than about, 50%, 40%, 30%, 20%, 15%, 10%, 9%, 8%, 7%, 6%, 5%, 4%, 3%, 2%, 1%, 0.5%, or 0% alpha-1,4, alpha-1,3 and/or alpha-1,2 glycosidic linkages. Typically, such linkages exist entirely, or almost entirely, as branch points from dextran.

The dextran portion of a dextran ether derivative herein can have alpha-1,2, alpha-1,3, and/or alpha-1,4 branches, for example. In some aspects, about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-35%, 2-30%, 2-25%, 2-20%, 2-15%, 2-10%, 3-35%, 3-30%, 3-25%, 3-20%, 3-15%, 3-10%, 5-35%, 5-30%, 5-25%, 5-20%, 5-15%, 5-10%, 7-13%, 8-12%, 9-11%, 10-35%, 10-30%, 10-25%, 10-20%, 10-15%, 12-20%, 12-18%, 14-20%, 14-18%, 15-35%, 15-30%, 15-25%, 15-20%, 15-18%, 15-17%, 17-23%, 18-22%, 19-21%, 20-35%, 20-30%, 20-25%, 35-45%, 37-43%, 38-42%, or 39-41% of all the glycosidic linkages of a branched dextran ether are alpha-1,2, alpha-1,3, and/or alpha-1,4 glycosidic branch linkages. Such branches typically are mostly (>90% or >95%), or all (100%), a single glucose monomer in length. In some aspects, dextran with alpha-1,2-branching can be produced enzymatically according to the procedures in U.S. Patent Appl. Publ. Nos. 2017/0218093 or 2018/0282385 (both incorporated herein by reference) where, for example, an alpha-1,2-branching enzyme such as GTFJ18T1 or GTF9905 can be added during or after the production of the dextran. In some aspects, any other enzyme known to produce alpha-1,2-branching can be used. Dextran with alpha-1,3-branching can be prepared, for example, as disclosed in Vuillemin et al. (2016, J. Biol Chem. 291:7687-7702) or Int. Patent Appl. Publ. No. WO2021/007264 or U.S. Patent Appl. Publ. No. 2022/0267745, which are incorporated herein by reference.

The dextran portion of a dextran ether derivative herein can have a DPw, DPn, or DP of about, at least about, or less than about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 85, 90, 95, 100, 105, 110, 150, 200, 250, 300, 400, 500, 1000, 1500, 2000, 2500, 3000, 4000, 5000, 6000, 8-20, 8-30, 8-100, 8-500, 3-4, 3-5, 3-6, 3-7, 3-8, 4-5, 4-6, 4-7, 4-8, 5-6, 5-7, 5-8, 6-7, 6-8, 7-8, 90-120, 95-120, 100-120, 105-120, 110-120, 115-120, 90-115, 95-115, 100-115, 105-115, 110-115, 90-110, 95-110, 100-110, 105-110, 90-105, 95-105, 100-105, 90-100, 95-100, 90-95, 85-95, 85-90, 5-100, 5-250, 5-500, 5-1000, 5-1500, 5-2000, 5-2500, 5-3000, 5-4000, 5-5000, 5-6000, 10-100, 10-250, 10-500, 10-1000, 10-1500, 10-2000, 10-2500, 10-3000, 10-4000, 10-5000, 10-6000, 25-100, 25-250, 25-500, 25-1000, 25-1500, 25-2000, 25-2500, 25-3000, 25-4000, 25-5000, 25-6000, 50-100, 50-250, 50-500, 50-1000, 50-1500, 50-2000, 50-2500, 50-3000, 50-4000, 50-5000, 50-6000, 100-100, 100-250, 100-400, 100-500, 100-1000, 100-1500, 100-2000, 100-2500, 100-3000, 100-4000, 100-5000, 100-6000, 250-500, 250-1000, 250-1500, 250-2000, 250-2500, 250-3000, 250-4000, 250-5000, 250-6000, 300-2800, 300-3000, 350-2800, 350-3000, 500-1000, 500-1500, 500-2000, 500-2500, 500-2800, 500-3000, 500-4000, 500-5000, 500-6000, 600-1550, 600-1850, 600-2000, 600-2500, 600-3000, 750-1000, 750-1250, 750-1500, 750-2000, 750-2500, 750-3000, 750-4000, 750-5000, 750-6000, 900-1250, 900-1500, 900-2000, 1000-1250, 1000-1400, 1000-1500, 1000-2000, 1000-2500, 1000-3000, 1000-4000, 1000-5000, 1000-6000, or 1100-1300, for example. The weight-average molecular weight (Mw) of the dextran portion of a dextran ether derivative in some aspects can be about, at least about, or less than about, 0.1, 0.125, 0.15, 0.175, 0.2, 0.24, 0.25, 0.5, 0.75, 1, 2, 3, 4, 5, 6, 7, 8, 9, 10, 20, 30, 40, 50, 60, 70, 80, 90, 100, 110, 120, 130, 140, 150, 160, 170, 180, 190, 200, 0.1-0.2, 0.125-0.175, 0.13-0.17, 0.135-0.165, 0.14-0.16, 0.145-0.155, 10-80, 20-70, 30-60, 40-50, 50-200, 60-200, 70-200, 80-200, 90-200, 100-200, 110-200, 120-200, 50-180, 60-180, 70-180, 80-180, 90-180, 100-180, 110-180, 120-180, 50-160, 60-160, 70-160, 80-160, 90-160, 100-160, 110-160, 120-160, 50-140, 60-140, 70-140, 80-140, 90-140, 100-140, 110-140, 120-140, 50-120, 60-120, 70-120, 80-120, 90-120, 90-110, 100-120, 110-120, 50-110, 60-110, 70-110, 80-110, 90-110, 100-110, 50-100, 60-100, 70-100, 80-100, 90-100, or 95-105 million Daltons. The Mw of the dextran portion of a dextran ether derivative in some aspects can be about, at least about, or less than about, 0.9, 1, 5, 7.5, 15, 10, 20, 30, 40, 50, 60, 70, 80, 90, 100, 150, 200, 250, 300, 400, 450, 500, 600, 700, 800, 900, 1000, 1250, 1500, 1750, 2000, 0.9-450, 1-500, 1-2000, 7.5-10, 7.5-15, 7.5-20, 10-350, 10-500, 10-400, 10-300, 10-200, 10-100, 10-50, 10-30, 15-25, 20-500, 20-400, 20-300, 20-200, 20-100, 20-50, 30-500, 30-400, 30-300, 30-200, 30-100, 30-50, 40-500, 40-400, 40-300, 40-200, 40-100, 40-60, 45-55, 40-50, 50-500, 50-400, 50-300, 50-350, 50-200, 80-300, 90-300, 100-500, 100-400, 100-300, 100-250, 100-200, 125-250, 150-250, 150-200, 175-200, 180-225, 180-200, 190-210, 200-500, 200-400, 200-300, or 290-310 kDa, for example. The molecular weight of dextran can be calculated, if desired, based on any of the foregoing dextran DPw, DPn, or DP values. Any of the forgoing DPw, DPn, DP, or Dalton values/ranges can characterize a dextran herein before, or after, it has optionally been branched (e.g., alpha-1,2 and/or alpha-1,3), for instance. In some aspects, any of the forgoing DPw, DPn, DP, or Dalton values or ranges can characterize a dextran ether derivative herein. The molecular weight of a dextran ether herein can be calculated, for example, based on any of the foregoing dextran DPw, DPn, DP, or Dalton values, further taking into account the ether's DoS and type of ether group(s).

The dextran portion of a dextran ether derivative herein can be as disclosed (e.g., molecular weight, linkage/branching profile, production method), for example, in U.S. Patent Appl. Publ. Nos. 2016/0122445, 2017/0218093, 2018/0282385, 2020/0165360, or 2019/0185893, which are each incorporated herein by reference. In some aspects, a dextran for ether derivatization can be one produced in a suitable reaction comprising glucosyltransferase (GTF) 0768 (SEQ ID NO:1 or 2 of US2016/0122445), GTF 8117, GTF 6831, or GTF 5604 (these latter three GTF enzymes are SEQ ID NOs:30, 32 and 33, respectively, of US2018/0282385), or a GTF comprising an amino acid sequence that is at least 90%, 91%, 92%, 93%, 94%, 95%, 96%, 97%, 98%, or 99% identical to the amino acid sequence of GTF 0768, GTF 8117, GTF 6831, or GTF 5604.

In some aspects, an ether derivative of an alpha-glucan of the present disclosure can have a degree of substitution (DoS) up to about 3.0 (e.g., 0.001 to 3.0) with at least one positively charged (cationic) organic group that is ether-linked to the alpha-glucan. The DoS can be about, at least about, or up to about, 0.001, 0.0025, 0.005, 0.01, 0.02, 0.025, 0.03, 0.04, 0.05, 0.06, 0.07, 0.075, 0.08, 0.09, 0.1, 0.15, 0.2, 0.25, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values), for example. Some examples of DoS ranges herein include 0.001-3.0, 0.001-2.5, 0.001-2.0, 0.001-1.5, 0.001-1.0, 0.001-0.5, 0.001-0.4, 0.001-0.3, 0.001-0.2, 0.001-0.175, 0.001-0.15, 0.001-0.125, 0.001-0.1, 0.01-3.0, 0.01-2.5, 0.01-2.0, 0.01-1.5, 0.01-1.0, 0.01-0.8, 0.01-0.5, 0.01-0.4, 0.01-0.3, 0.01-0.2, 0.01-0.175, 0.01-0.15, 0.01-0.125, 0.01-0.1, 0.03-0.7, 0.04-0.6, 0.05-3.0, 0.05-2.5, 0.05-2.0, 0.05-1.5, 0.05-1.0, 0.05-0.5, 0.05-0.8, 0.05-0.4, 0.05-0.3, 0.05-0.2, 0.05-0.175, 0.05-0.15, 0.05-0.125, 0.05-0.1, 0.1-3.0, 0.1-2.5, 0.1-2.0, 0.1-1.5, 0.1-1.0, 0.1-0.8, 0.1-0.5, 0.1-0.4, 0.1-0.3, 0.1-0.2, 0.1-0.175, 0.1-0.15 and 0.1-0.125.

Since there are at most three hydroxyl groups in a glucose monomeric unit of an alpha-glucan, the overall DoS of an alpha-glucan ether derivative can be no higher than 3.0. It would be understood by those skilled in the art that, since an alpha-glucan ether derivative as presently disclosed has a DoS with at least one type of positively charged organic group in ether linkage (e.g., between about 0.001 to about 3.0), all the substituents of an alpha-glucan ether derivative cannot only be hydroxyl.

An ether derivative of an alpha-glucan of the present disclosure can be substituted with at least one positively charged organic group herein that is ether-linked to the alpha-glucan. A positively charged organic group can be, for example, any of those disclosed in U.S. Patent Appl. Publ. Nos. 2016/0311935, 2018/0237816, or 2020/0002646, or International Pat. Appl. Publ. No. WO2021/257786, which are incorporated herein by reference. A positively charged organic group can comprise a substituted ammonium group, for example. Examples of substituted ammonium groups are primary, secondary, tertiary and quaternary ammonium groups, such as can be represented by Structures I and II. An ammonium group can be substituted with alkyl group(s) and/or aryl group(s), for example. There can be one, two, or three types of alkyl and/or aryl groups in some aspects of a substituted ammonium group. An alkyl group of a substituted ammonium group herein can be a C₁-C₃₀ alkyl group, for example, such as a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, C₂₅, C₂₆, C₂₇, C₂₈, C₂₉, or C₃₀ group, each alkyl group can be the same or different in aspects with two or three alkyl substitutions. An alkyl group can be C₁-C₂₄, C₁-C₁₈, C₆-C₂₀, C₁₀-C₁₆, or C₁-C₄ in some aspects. An aryl group can be a C₆, C₆-C₂₄, C₁₂-C₂₄, or C₆-C₁₈ aryl group, for example, that is optionally substituted with one or more alkyl substituents (e.g., any alkyl group disclosed herein).

A secondary ammonium alpha-glucan ether herein can comprise a monoalkylammonium group in some aspects (e.g., based on Structure I). A secondary ammonium alpha-glucan ether can be a monoalkylammonium alpha-glucan ether in some aspects, such as a monomethyl-, monoethyl-, monopropyl-, monobutyl-, monopentyl-, monohexyl-, monoheptyl-, monooctyl-, monononyl-, monodecyl-, monoundecyl-, monododecyl-, monotridecyl-, monotetradecyl-, monopentadecyl-, monohexadecyl-, monoheptadecyl-, or monooctadecyl-ammonium alpha-glucan ether. These alpha-glucan ethers can also be referred to as methyl-, ethyl-, propyl-, butyl-, pentyl-, hexyl-, heptyl-, octyl-, nonyl-, decyl-, undecyl-, dodecyl-, tridecyl-, tetradecyl-, pentadecyl-, hexadecyl-, heptadecyl-, or octadecyl-ammonium alpha-glucan ethers, respectively.

A tertiary ammonium alpha-glucan ether herein can comprise a dialkylammonium group in some aspects (e.g., based on Structure I). A tertiary ammonium alpha-glucan ether can be a dialkylammonium alpha-glucan ether in some aspects, such as a dimethyl-, diethyl-, dipropyl-, dibutyl-, dipentyl-, dihexyl-, diheptyl-, dioctyl-, dinonyl-, didecyl-, diundecyl-, didodecyl-, ditridecyl-, ditetradecyl-, dipentadecyl-, dihexadecyl-, diheptadecyl-, or dioctadecyl-ammonium alpha-glucan ether.

A quaternary ammonium alpha-glucan ether herein can comprise a trialkylammonium group in some aspects (e.g., based on Structure I). A quaternary ammonium alpha-glucan ether compound can be a trialkylammonium alpha-glucan ether in some aspects, such as trimethyl-, triethyl-, tripropyl-, tributyl-, tripentyl-, trihexyl-, triheptyl-, trioctyl-, trinonyl-, tridecyl-, triundecyl-, tridodecyl-, tritridecyl-, tritetradecyl-, tripentadecyl-, trihexadecyl-, triheptadecyl-, or trioctadecyl-ammonium alpha-glucan ether.

In some aspects, a positively charged organic group can comprise a C₄ to C₂₀ alkyl group. A C₄ to C₂₀ alkyl group can be any one of a C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl group, for example. In some aspects, an alkyl group can be a C₁₀ to C₁₄ alkyl group, meaning that the alkyl group can be any one of a C₁₀, C₁₁, C₁₂, C₁₃, or C₁₄ alkyl group. Additional examples include an alkyl group that is a C₆ to C₁₈, C₈ to C₁₈, C₁₀ to C₁₈, C₆ to C₁₆, C₈ to C₁₆, C₁₀ to C₁₆, C₆ to C₁₄, C₈ to C₁₄, C₁₀ to C₁₄, C₆ to C₁₂, C₈ to C₁₂, or C₁₀ to C₁₂ alkyl group. By disclosing a C₁₂ alkyl group, for example, it is meant that the alkyl group is twelve carbons in length and is saturated (i.e., —CH₂CH₂CH₂CH₂CH₂CH₂CH₂CH₂CH₂CH₂CH₂CH₃); this standard meaning applies, accordingly, to other alkyl groups disclosed herein.

In some aspects, a positively charged organic group can comprise a C₄ to C₂₀ alkylene group (e.g., of any length as disclosed herein for an alkyl group). An alkylene group can comprise one, two, three, or more double-bonds, for example. An alkylene group in some aspects can comprise one or more double-bonds spanning carbons (i) 5 and 6, (ii) 6 and 7, (iii) 8 and 9, (iv) 9 and 10, (v) 11 and 12, (vi) 12 and 13, (vii) 14 and 15, and/or (viii) 15 and 16 of the alkylene group, where carbon number is counted starting from the carbon directly linked to the positively charged group (e.g., carbon-1 is linked to the nitrogen of a substituted ammonium group herein). Some combinations of double-bonds of an alkylene group include: (iv) and (vi); (iv), (vi) and (vii); and (i), (iii), (v) and (vii) (with reference to the foregoing list). While a double-bond herein of an alkylene group can be in a cis or trans orientation, it typically is in the cis orientation. An alkylene group can be derived (derivable) from a fatty acid (e.g., caproic acid, caprylic acid, capric acid, lauric acid, myristic acid, palmitic acid, stearic acid, arachidic acid, oleic acid, linoleic acid, arachidonic acid), or an acyl group (e.g., corresponding to any fatty acid herein) of a lipid (e.g., a mono-, di-, or tri-glyceride), for example.

In some aspects, a substituted ammonium group is a tertiary ammonium group in which, with reference to Structure I and/or II, R₂ is a hydrogen atom, R₃ is a methyl, ethyl, propyl, or butyl, and R₄ is any C₄ to C₂₀ alkyl or alkylene group as described above. In some aspects, a substituted ammonium group is a quaternary ammonium group in which, with reference to Structure I and/or II, R₂ and R₃ are each independently a methyl, ethyl, propyl, or butyl (e.g., both R₂ and R₃ are methyl, or are both ethyl), and R₄ is any C₄ to C₂₀ alkyl or alkylene group as described above (e.g., a C₁₂ alkyl). A tertiary or quaternary ammonium group in some aspects comprises Structure II, and has any of the foregoing R₂, R₃ and R₄ assignments.

In some aspects, at least one of the groups of a substituted ammonium group comprises one carbon, or a chain of carbons (e.g., up to 30), in ether linkage to an alpha-glucan. A carbon chain in this context can be linear, for example. Such a carbon or carbon chain can be represented by —CH₂—, —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, —CH₂(CH₂)₈CH₂—, —CH₂(CH₂)₉CH₂—, or —CH₂(CH₂)₁₀CH₂—, for example. In some aspects, a carbon chain in this context can be branched, such as by being substituted with one or more alkyl groups (e.g., any as disclosed above such as methyl, ethyl, propyl, or butyl). The point(s) of substitution can be anywhere along the carbon chain. Examples of branched carbon chains include —CH(CH₃)CH₂—, —CH(CH₃)CH₂CH₂—, —CH₂CH(CH₃)CH₂—, —CH(CH₂CH₃)CH₂—, —CH(CH₂CH₃)CH₂CH₂—, —CH₂CH(CH₂CH₃)CH₂—, —CH(CH₂CH₂CH₃)CH₂—, —CH(CH₂CH₂CH₃)CH₂CH₂— and —CH₂CH(CH₂CH₂CH₃)CH₂—; longer branched carbon chains can also be used, if desired. In some aspects, a chain of one or more carbons (e.g., any of the above linear or branched chains) is further substituted with one or more hydroxyl groups. Examples of hydroxy- or dihydroxy (diol)-substituted chains include —CH(OH)—, —CH(OH)CH₂—, —C(OH)₂CH₂—, —CH₂CH(OH)CH₂—, —CH(OH)CH₂CH₂—, —CH(OH)CH(OH)CH₂—, —CH₂CH₂CH(OH)CH₂—, —CH₂CH(OH)CH₂CH₂—, —CH(OH)CH₂CH₂CH₂—, —CH₂CH(OH)CH(OH)CH₂—, —CH(OH)CH(OH)CH₂CH₂— and —CH(OH)CH₂CH(OH)CH₂—. In each of the foregoing examples, the first carbon atom of the chain is ether-linked to a glucose monomer of the alpha-glucan, and the last carbon atom of the chain is linked to a positively charged group (e.g., a substituted ammonium group as disclosed herein). One or more positively charged organic groups in some aspects can comprise trimethylammonium hydroxypropyl groups (Structure II, when each of R₂, R₃ and R₄ is a methyl group).

In aspects in which a carbon chain of a positively charged organic group has a substitution in addition to a substitution with a positively charged group, such additional substitution can be with one or more hydroxyl groups, oxygen atoms (thereby forming an aldehyde or ketone group), alkyl groups (e.g., methyl, ethyl, propyl, butyl), and/or additional positively charged groups, for example. A positively charged group is typically bonded to the terminal carbon atom of the carbon chain. A positively charged group can also comprise imidazoline ring-containing compounds in some aspects.

A counter ion for a positively charged organic group herein can be any suitable anion, such as an acetate, borate, bromate, bromide, carbonate, chlorate, chloride, chlorite, dihydrogen phosphate, fluoride, hydrogen carbonate, hydrogen phosphate, hydrogen sulfate, hydrogen sulfide, hydrogen sulfite, hydroxide, hypochlorite, iodate, iodide, nitrate, nitride, nitrite, oxalate, oxide, perchlorate, permanganate, phosphate, phosphide, phosphite, silicate, stannate, stannite, sulfate, sulfide, sulfite, tartrate, or thiocyanate anion.

An alpha-glucan ether in some aspects can contain one type of etherified positively charge organic group. Examples of such a positively charge organic group are as disclosed herein. Optionally, an alpha-glucan ether compound having a single type of etherified positively charge organic group can be characterized as a monoether. In some aspects, an alpha-glucan ether can contain two or more different types of etherified positively charge organic groups (i.e., a mixed ether). An alpha-glucan ether in some aspects has no other types of organic groups derivatized to the alpha-glucan (e.g., a hydrophobic group that, for example, is ether- or ester-linked to the alpha-glucan).

A liquid composition in some aspects can comprise one or more impurities/byproducts. Thus, in some aspects, a product produced using a liquid composition of the present disclosure as an ingredient can also comprise the one or more impurities/byproducts. Herein, unless otherwise stated, the term “impurities” will refer to both impurities (e.g., compounds originating from inadvertent introduction to a liquid composition herein or precursor of the liquid composition such as an etherification reaction composition [e.g., an impurity present in an etherification agent preparation]; e.g., compounds originating from purposeful introduction to a precursor of a liquid composition, such as a preservative [e.g., sodium benzoate, 2-methyl-4-isothiazolin-3-one, 5-chloro-2-methyl-4-isothiazolin-3-one, sorbate, benzisothiazolinone], protein/enzyme [e.g., GTF/sucrase], salt [NaCl, Na₂SO₄], buffer, and/or reagent [e.g., etherification agent]) and byproducts (e.g., compounds produced as secondary non-targeted products in a glucosyltransferase reaction[s] used to produce an alpha-glucan herein such as alpha-1,6-glucan enzymatic synthesis or enzymatic branching [e.g., alpha-1,2 and/or alpha-1,3 branching], e.g., compounds produced as secondary non-targeted products in an etherification reaction composition). In some aspects, an impurity or byproduct may be converted to another form of impurity or byproduct through the action of an enzymatic branching reaction (e.g., alpha-1,2 and/or alpha-1,3 branching), cationic etherification reaction, and/or other process herein (e.g., heating/cooling, pH modification, reaction with other compounds). While an etherification reaction used to produce a cationic alpha-glucan ether herein typically is purified to remove impurities completely and/or to render them as undetectable, one or more of such compounds can be present in some aspects.

A byproduct of a glucosyltransferase (GTF) reaction in some aspects can be glucose, leucrose, and/or one or more alpha-gluco-oligosaccharides (GOS, e.g., DP2-DP7). Though technically not GTF reaction byproducts per se, the GTF reaction coproduct, fructose, and/or any unreacted sucrose substrate can also be impurities in some aspects. Further examples of GTF reaction byproducts/impurities herein can be as disclosed in U.S. Patent Appl. Publ. Nos. 2017/0218093, 2018/0282385, 2016/0122445, 2020/0165360, 2019/0185893, or 2022/0267745, or Int. Patent Appl. Publ. Nos. WO2021/007264 or WO2021/257786, or Vuillemin et al. (2016, J. Biol Chem. 291:7687-7702), which are incorporated herein by reference. An impurity in some cases can a compound that is produced when any of the foregoing GTF reaction byproducts/impurities is/are modified, if possible, by a branching GTF (e.g., alpha-1,2 and/or alpha-1,3 branching). In some aspects, an impurity can be any of the foregoing species that is carried over into an etherification reaction and modified therein (e.g., etherified such as presently disclosed).

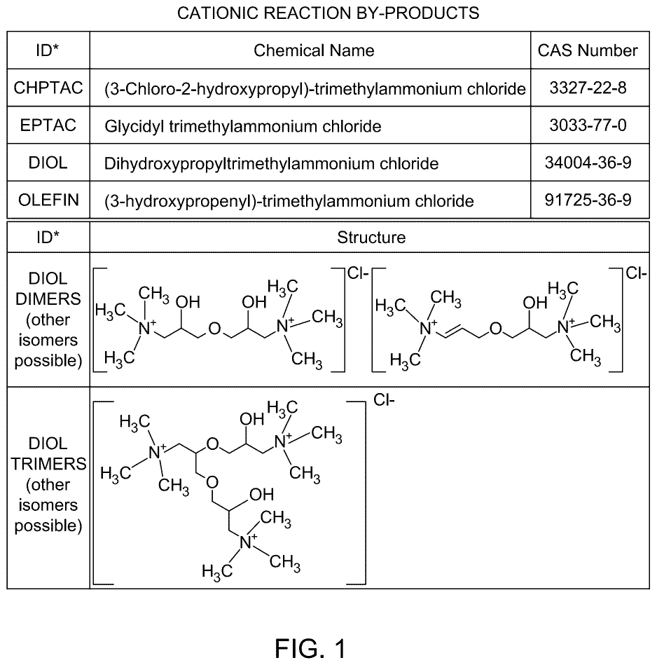

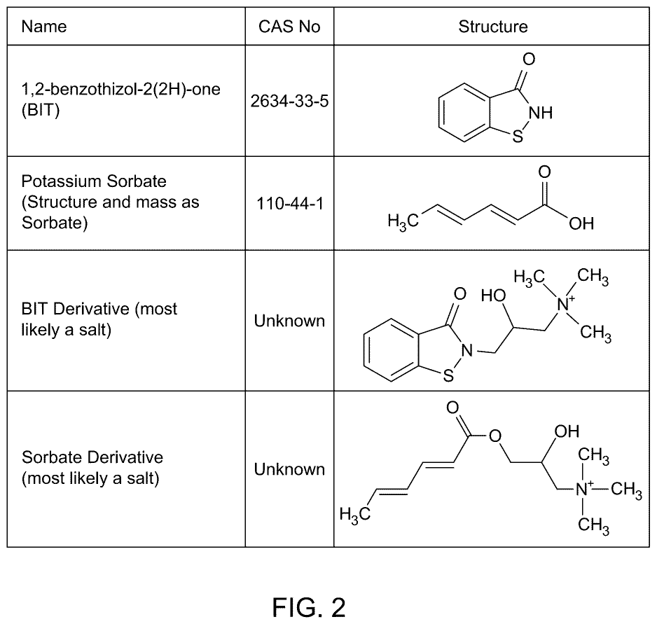

An impurity in some aspects can be one or more of those presented in any of FIGS. 1-2, for example, and/or as disclosed in Kavaliauskaite et al. (2008, Carbohydr. Polym. 73:665-675, incorporated herein by reference). For example, an impurity can be as disclosed in Scheme 1 of Kavaliauskaite et al. (ibid.) or FIGS. 1-2, and/or be a product of such an impurity as reacted with an alpha-glucan synthesis reaction-based species (e.g., such a species may be a byproduct from producing alpha-1,6-glucan and/or glycosidic branches therefrom, such as disclosed herein).

In some aspects, one or more impurities, such as any of those disclosed herein, can be present at about, or less than about, 1000, 900, 800, 700, 600, 500, 400, 300, 200, 100, 50, 25, 10, 5, 2.5, 1.0, 0.5, 0.25, 0.1, 0.05, 0.025, or 0.01 parts-per-million (ppm).

Some aspects herein regard a method/process of producing a liquid composition as presently disclosed. Such a method can comprise:
(a) providing an aqueous composition comprising at least one cationic alpha-glucan ether derivative (e.g., any as disclosed herein),
(b) mixing, into the aqueous composition, at least one organic solvent herein (e.g., a suitable amount of organic solvent to achieve a desired concentration of the organic solvent in the final liquid composition produced after this step or after optional step [c]), and
(c) optionally concentrating (removing water, such as by evaporation) the at least one cationic alpha-glucan ether derivative and the at least one organic solvent in the aqueous composition following step (b). Step (c) can be necessary in some aspects to reach particular concentration(s) of the cationic alpha-glucan ether derivative and/or organic solvent.

An aqueous composition provided in step (a) can be, for example, an etherification reaction composition in which a cationic alpha-glucan ether derivative was produced. Typically, an etherification reaction composition entered in step (a) of the method has been terminated/quenched and/or neutralized. An etherification reaction herein can be as described in the below Examples, or as disclosed in International Pat. Appl. Publ. No. WO2021/257786 (incorporated herein by reference), for example. Step (a) in some aspects can comprise subjecting an etherification reaction composition to one, two, three or more rounds of a purification process to increase the purity of the cationic alpha-glucan ether derivative in the aqueous composition. Such purification can be performed by a process comprising diafiltration (e.g., ultrafiltration or nanofiltration) and/or dialysis, for example.

Some aspects of the present disclosure regard a product comprising a liquid composition herein that comprises (i) at least one organic solvent, (ii) at least one cationic alpha-glucan ether derivative, and (iii) water, typically wherein the liquid composition was used as an ingredient/component in producing the product. A product herein made with the liquid composition as an ingredient typically is in liquid form, or at least contains a liquid component. However, in some aspects, a dry or non-aqueous product can be produced in part using a liquid composition herein.

In some aspects, a product can comprise about, at least about, or less than about, 0.01, 0.05, 0.1, 0.2, 0.25, 0.3, 0.4, 0.5, 0.6, 0.7, 0.75, 0.8, 0.9, 1.0, 1.2, 1.25, 1.4, 1.5, 1.6, 1.75, 1.8, 2.0, 2.25, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 99.5 wt % or w/v % of a cationic alpha-glucan ether derivative herein. A product can comprise a range between any two of these wt % or w/v % values (e.g., 5-50, 5-45, 5-40, 5-35, 5-30, 5-25, 5-20, 5-15, 5-10, 0.1-1.0, 0.1-0.75, 0.1-0.5, 0.1-0.4, 0.1-0.3, 0.2-1.0, 0.2-0.75, 0.2-0.5, 0.2-0.4, 0.2-0.3, 0.3-1.0, 0.3-0.75, 0.3-0.5, or 0.3-0.4 wt % or w/v %), for example. A product can comprise a liquid component that is water (i.e., the organic solvent of the liquid composition ingredient has been removed), or that comprises about, or at least about, 10, 20, 30, 40, 50, 60, 70, 80, 90, 95, 98, or 99 wt % water, for example. A liquid component can be in the form of a solution, or a mixture such as a colloidal dispersion or emulsion, for example.

A liquid component of a product herein, or a liquid composition of the present disclosure, can have a viscosity of about, at least about, or less than about, 1, 5, 10, 100, 200, 300, 400, 500, 600, 700, 1000, 2000, 3000, 4000, 5000, 6000, 7000, 8000, 9000, 10000, 15000, 1-300, 10-300, 25-300, 50-300, 1-250, 10-250, 25-250, 50-250, 1-200, 10-200, 25-200, 50-200, 1-150, 10-150, 25-150, 50-150, 1-100, 10-100, 25-100, or 50-100 centipoise (cps), for example. Viscosity can be as measured with a liquid herein at any temperature between about 3 °C to about 80 °C, for example (e.g., 4-30 °C, 15-30 °C, 15-25 °C), or any particular temperature disclosed herein for a liquid composition. Viscosity typically is as measured at atmospheric pressure (about 760 torr) or a pressure that is ±10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, 1.0-1000, or 1.0-100 s⁻¹ (1/s), or about 5, 10, 20, 25, 50, 100, 200, or 250 rpm (revolutions per minute), for example.

A product in some instances be non-aqueous (e.g., a dry composition). Examples of such embodiments include powders, granules, microcapsules, flakes, or any other form of particulate matter. Other examples include larger compositions such as pellets, bars, kernels, beads, tablets, sticks, or other agglomerates, or ointment or lotion (or any other form herein of a non-aqueous or dry composition). A non-aqueous or dry product typically has about, or no more than about, 3, 2, 1.0, 0.5, 0.25, 0.10, 0.05, or 0.01 wt % water comprised therein, but in some cases can have about 10-12 or 10-15 wt % water. In some aspects (e.g., those directed to a laundry or dish washing detergent), a dry product can be provided in a sachet or pouch.

A product herein can, in some aspects, be a detergent product. Examples of such products are disclosed herein as detergents for dishwashing and detergents for fabric care.

A product herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous product herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A product herein can optionally contain one or more enzymes (active enzymes). Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases, and amylases. If an enzyme(s) is included, it may be comprised in a product herein at about 0.0001-0.1 wt % (e.g., 0.01-0.03 wt %) active enzyme (e.g., calculated as pure enzyme protein), for example. In fabric care or automatic dishwashing applications, an enzyme herein (e.g., any of the above such as cellulase, protease, amylase, and/or lipase) can be present in an aqueous composition in which a fabric or dish is treated (e.g., wash liquor, grey water) at a concentration that is minimally about 0.01-0.1 ppm total enzyme protein, or about 0.1-10 ppb total enzyme protein (e.g., less than 1 ppm), to maximally about 100, 200, 500, 1000, 2000, 3000, 4000, or 5000 ppm total enzyme protein, for example.

A cationic alpha-glucan ether derivative and/or a product comprising such a derivative is biodegradable in some aspects. Such biodegradability can be, for example, as determined by the Carbon Dioxide Evolution Test Method (OECD Guideline 301B, incorporated herein by reference), to be about, at least about, or at most about, 5%, 10%, 15%, 20%, 25%, 30%, 35%, 40%, 45%, 50%, 55%, 60%, 65%, 70%, 75%, 80%, 85%, 90%, 5-60%, 5-80%, 5-90%, 40-70%, 50-70%, 60-70%, 40-75%, 50-75%, 60-75%, 70-75%, 40-80%, 50-80%, 60-80%, 70-80%, 40-85%, 50-85%, 60-85%, 70-85%, 40-90%, 50-90%, 60-90%, or 70-90%, or any value between 5% and 90%, after 15, 30, 45, 60, 75, or 90 days of testing.

A product as presently disclosed (e.g., a product comprising a liquid composition herein that comprises [i] at least one organic solvent, [ii] at least one cationic alpha-glucan ether derivative, and [iii] water, typically wherein the liquid composition was used as an ingredient/component in producing the product) can be in the form of a household care (home care) product, personal care product, industrial product, medical product, or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or Int. Patent Appl. Publ. No. WO2016/133734, which are all incorporated herein by reference. In some aspects, a product can comprise at least one component/ingredient of a household care (home care) product, personal care product, industrial product, medical product, or pharmaceutical product as disclosed in any of the foregoing publications and/or as presently disclosed.

A product in some aspects is believed to be useful for providing one or more of the following physical properties to a personal care product, pharmaceutical product, household care product, or industrial product: thickening, freeze/thaw stability, lubricity, moisture retention and release, texture, consistency, shape retention, emulsification, binding, suspension, dispersion, gelation, or reduced mineral hardness, for example.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect.

In some aspects, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil. A skin care product can be an ointment, lotion, or sanitizer (e.g., hand sanitizer) in some aspects.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a skin cleanser, soap, skin-washing product, or related product, or any product that can be applied and then rinsed from skin. Some benefits of such a product can be better rinsability from skin after application (e.g., lathering on skin), and/or enhanced skin-fell, such as a reduced sensation of skin roughness and/or over-dryness from using the product (e.g., after rinsing the product from the skin and optionally towel-drying the skin).

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse (e.g., hair styling mousse), hair spray (e.g., hair styling spray), and styling gel (e.g., hair styling gel). A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A product in some aspects can be a hair care composition such as a hair styling or hair setting composition (e.g., hair spray, hair gel or lotion, hair mousse/foam) (e.g., aerosol hair spray, non-aerosol pump-spray, spritze, foam, creme, paste, non-runny gel, mousse, pomade, lacquer, hair wax). A hair styling/setting composition/formulation that can be adapted to include at least one alpha-glucan ether derivative herein can be as disclosed in, for example, US20090074697, WO1999048462, US20130068849, JPH0454116A, U.S. Pat. No. 5,304,368, AU667246B2, U.S. Pat. Nos. 5,413,775, 5,441,728, 5,939,058, JP2001302458A, U.S. Pat. No. 6,346,234, US20020085988, U.S. Pat. No. 7,169,380, US20090060858, US20090326151, US20160008257, WO2020164769, or US20110217256, all of which are incorporated herein by reference. A hair care composition such as a hair styling/setting composition can comprise one or more ingredients/additives as disclosed in any of the foregoing references, and/or one or more of a fragrance/perfume, aroma therapy essence, herb, infusion, antimicrobial, stimulant (e.g., caffeine), essential oil, hair coloring, dying or tinting agent, anti-gray agent, anti-foam agent, sunscreen/UV-blocker (e.g., benzophenone-4), vitamin, antioxidant, surfactant or other wetting agent, mica, silica, metal flakes or other glitter-effect material, conditioning agent (e.g., a volatile or non-volatile silicone fluid), anti-static agent, opacifier, detackifying agent, penetrant, preservative (e.g., phenoxyethanol, ethylhexylglycerin, benzoate, diazolidinyl urea, iodopropynyl butylcarbamate), emollient (e.g., panthenol, isopropyl myristate), rheology-modifying or thickening polymer (e.g., acrylates/methacrylamide copolymer, polyacrylic acid [e.g., CARBOMER]), emulsified oil phase, petrolatum, fatty alcohols, diols and polyols, emulsifier (e.g., PEG-40 hydrogenated castor oil, Oleth-20), humectant (e.g., glycerin, caprylyl glycol), silicone derivative, protein, amino acid (e.g., isoleucine), conditioner, chelant (e.g., EDTA), solvent (e.g., see below), monosaccharide (e.g., dextrose), disaccharide, oligosaccharide, pH-stabilizing compound (e.g., aminomethyl propanol), film former (e.g., acrylates/hydroxyester acrylate copolymer, polyvinylpyrrolidone/vinyl acetate copolymer, triethyl acetate), aerosol propellant (e.g., C₃-C₅ alkane such as propane, isobutane, or n-butane, monoalkyl ether, dialkyl ether such as di(C₁-C₄ alkyl) ether [e.g., dimethyl ether]), and/or any other suitable material herein. An alpha-glucan ether derivative as used in a hair styling/setting composition in some aspects can function as a hair fixing/styling agent (typically non-permanent hair fixing, but durable), and optionally is the only hair fixing agent in the composition. Optional additional hair fixing/styling agents herein include PVP (polyvinylpyrrolidone), octylacrylamide/acrylates/butylaminoethyl methacrylate copolymer, vinyl caprolactam/PVP/dimethylaminoethyl methacrylate copolymer, AMPHOMER, or any film former such as listed above.

The total content of one or more alpha-glucan ether derivatives in a hair care composition such as a hair styling/setting composition herein can be about, at least about, or less than about, 0.5, 1, 2, 3, 4, 5, 6, 7, 8, 9, 10, 11, 12, 13, 14, 15, 0.5-15, 0.5-10, 0.5-5, 0.5-2, 1-15, 1-10, 1-5, 1-2, 2.5-7.5, 3-7, or 4-6 wt %, for example. A hair styling/setting composition can comprise a solvent comprising water and optionally a water-miscible (typically polar) organic compound (e.g., liquid or gas) such as an alcohol (e.g., ethanol, propanol, isopropanol, n-butanol, iso-butanol, tert-butanol), an alkylene glycol alkyl ether, and/or a monoalkyl or dialkyl ether (e.g., dimethyl ether), for example. If an organic compound is included, it can constitute about 10%, 20%, 30%, 40%, 50%, or 60% by weight or volume of the solvent (balance is water), for example. The amount of solvent in a hair styling/setting composition herein can be about 50-90, 60-90, 70-90, 80-90, 50-95, 60-95, 70-95, 80-95, or 90-95 wt %, for example.

An example of a hair styling gel formulation herein can comprise about 90-95 wt % (e.g., ~92 wt %) solvent (e.g., water), 0.3-1.0 wt % (e.g., ~0.5 wt %) thickener (e.g., polyacrylic acid), 0.1-0.3 wt % (e.g., ~0.2 wt %) chelant (e.g., EDTA) (optional), 0.2-1.0 wt % (e.g., ~0.5 wt %) humectant (e.g., glycerin), 0.01-0.05 wt % (e.g., ~0.02 wt %) UV-blocker (e.g., benzophenone-4) (optional), 0.05-0.3 wt % (e.g., ~0.1 wt %) preservative (e.g., diazolidinyl urea) (optional), 0.5-1.2 wt % (e.g., ~0.8 wt %) emulsifier (e.g., Oleth-20), 0.1-0.3 wt % (e.g., ~0.2 wt %) fragrance/perfume (optional), 0.2-1.0 wt % (e.g., ~0.5 wt %) pH-stabilizing compound (e.g., aminomethyl propanol), and 3-7 wt % (e.g., ~5 wt %) alpha-glucan ether derivative herein (e.g., as a hair fixing/styling agent).

An example of a hair styling spray formulation herein can comprise about 0.2-1.0 wt % (e.g., +0.5 wt %) pH-stabilizing compound (e.g., aminomethyl propanol), 0.1-0.3 wt % (e.g., ~0.2 wt %) fragrance/perfume (optional), 0.05-0.12 wt % (e.g., ~0.08 wt %) surfactant (e.g., ethoxylated dimethicone polyol), 0.05-0.12 wt % (e.g., ~0.08 wt %) conditioner (e.g., cyclomethicone) (optional), 0.05-0.3 wt % (e.g., ~0.2 wt %) preservative (e.g., sodium benzoate) (optional), 15-20 wt % (e.g., ~17 wt %) water, 30-40 wt % (e.g., ~65 wt %) alcohol (e.g., ethanol), 40-60 wt % (e.g., ~45 wt %) propellant (e.g., dimethyl ether, or a ~2:1 mix of dimethyl ether to C₃-C₅ alkane [e.g., mix of propane and isobutane]), and 2-4 wt % (e.g., ~2.75 wt %) alpha-glucan ether derivative herein (e.g., as a hair fixing/styling agent).

Some aspects of the present disclosure regard hair that has been treated with a hair care composition herein (e.g., hair styling/setting composition, shampoo, or conditioner). For example, hair can comprise an alpha-glucan ether derivative on its surface, such as in a film/coating of the hair, and/or adsorbed or otherwise deposited on the hair surface, optionally, one or more other ingredients of a hair care composition herein can also be present. In some aspects, hair as presently disclosed, such as hair with a coating comprising an alpha-glucan ether, does not exhibit flaking to the naked eye (i.e., little or no noticeable flaking). In some aspects, hair as presently disclosed that has been treated with a hair care composition herein such as a shampoo or conditioner (and thus typically coated with an alpha-glucan ether), typically followed by water rinsing, requires a lower amount of energy (e.g., lessened by about, or at least about, 5%, 6%, 7%, 8%, 9%, 10%, 11%, 12%, 13%, 14%, 15%, 16%, 17%, 18%, 19%, 20%) to be combed, either when the hair is wet or dry (e.g., as compared to hair otherwise treated in the same manner but with a hair care composition that does not comprise an alpha-glucan ether derivative herein and that optionally instead comprises an incumbent/conventional hair treatment/conditioning polymer such as polyquaternium-10, guar hydroxypropyltrimonium chloride, polyquaternium-7, hydroxypropyl guar hydroxypropyltrimonium chloride). Hair combing energy can be measured as disclosed in U.S. Pat. Appl. Publ. No. 2014/0271504 (incorporated herein by reference) or as described in the below Examples, for example.

Various examples of personal care formulations comprising at least one alpha-glucan ether derivative as presently disclosed are disclosed below (1-8).
(1) A hair conditioner composition comprising: cetyl alcohol (1-3%), isopropyl myristate (1-3%), hydroxyethyl cellulose (Natrosol® 250 HHR, 0.1-1%), alpha-glucan ether derivative (0.1-2%), potassium salt (0.1-0.5%), Germaben® II preservative (0.5%, available from International Specialty Products), and the balance being water.
(2) A hair shampoo composition comprising: 5-20% sodium laureth sulfate (SLES), 1-2 wt % cocamidopropyl betaine, 1-2 wt % sodium chloride, 0.1-2% alpha-glucan ether derivative, preservative (0.1-0.5%), and the balance being water.
(3) A hair shampoo composition according to Table 4 herein comprising an alpha-glucan ether derivative, but where the amount of each ingredient (cationic alpha-glucan ether, cocamidopropyl betaine, sodium C14-16 olefin sulfonate, phenoxyethanol, sodium chloride, citric acid anhydrous, disodium EDTA, water) is within 5%, 10%, 15%, or 20% of the amount listed in Table 4.
(4) A hair shampoo composition according to Table 6 herein comprising an alpha-glucan ether derivative, but where the amount of each ingredient (cationic alpha-glucan ether, cocamidopropyl betaine, sodium C14-16 olefin sulfonate, phenoxyethanol, sodium chloride, citric acid anhydrous, disodium EDTA, water) is within 5%, 10%, 15%, or 20% of the amount listed in Table 6.
(5) A hair shampoo composition according to Table 8 herein comprising an alpha-glucan ether derivative, but where the amount of each ingredient (cationic alpha-glucan ether, cocamidopropyl betaine, sodium laureth sulfate, dimethicone, phenoxyethanol, sodium chloride, citric acid anhydrous, disodium EDTA, water) is within 5%, 10%, 15%, or 20% of the amount listed in Table 8.
(6) A skin lotion composition comprising: 1-5% glycerin, 1-5% glycol stearate, 1-5% stearic acid, 1-5% mineral oil, 0.5-1% acetylated lanolin (Lipolan® 98), 0.1-0.5 cetyl alcohol, 0.2-1% triethanolamine, 0.1-1 wt % Germaben® II preservative, 0.5-2 wt % alpha-glucan ether derivative, and the balance being water.
(7) A skin cleanser or soap composition according to Table 14 herein comprising an alpha-glucan ether derivative, but where the amount of each ingredient (cationic alpha-glucan ether, cocamidopropyl betaine, caprylyl/capryl glucoside, sodium benzoate, citric acid, water) is within 5%, 10%, 15%, or 20% of the amount listed in Table 14.
(8) A skin cleanser or soap composition according to Table 15 herein comprising an alpha-glucan ether derivative, but where the amount of each ingredient (cationic alpha-glucan ether, cocamidopropyl betaine, sodium laureth sulfate, potassium laurate, phenoxyethanol, sodium chloride, water) is within 5%, 10%, 15%, or 20% of the amount listed in Table 15.

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A composition herein can also be used, for example, in capsules, tablets, tablet coatings, and as excipients for medicaments and drugs.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; latex; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example. In some aspects, a composition herein is comprised in a fluid as a viscosity modifier and/or friction reducer; such uses include downhole operations/fluids (e.g., in hydraulic fracturing and enhanced oil recovery), for example.

Some aspects herein regard (i) salt water such as seawater, or (ii) an aqueous solution having about 2.0, 2.25, 2.5, 2.75, 3.0, 3.25. 3.5, 3.75, 4.0, 2.5-4.0, 2.75-4.0, 3.0-4.0, 2.5-3.5, 2.75-3.5, 3.0-3.5, 3.0-4.0, or 3.0-3.5 wt % of one or a combination of salts (e.g., including at least NaCl), having at least one aqueous-soluble alpha-glucan ether derivative as presently disclosed. The concentration of an alpha-glucan ether derivative in such water of (i) or (ii) can be about, at least about, or below about, 0.1, 0.2, 0.3, 0.4, 0.5, 0.6, 0.7, 0.8, 0.9, 1.0, 0.1-0.6, 0.1-0.5, 0.1-0.4, 0.1-0.3, or 0.1-0.2 wt %, for example. Despite the relatively high salt concentration in such aqueous compositions, it is contemplated that an alpha-glucan ether derivative in some aspects can remain completely or mostly in solution and provide viscosity. Such a solution of (i) or (ii) as viscosity-modified by an alpha-glucan ether derivative herein can be as it is used within a system that utilizes such a solution (e.g., any herein, such as a downhole operation).

Yet, in some aspects, an alpha-glucan ether derivative does not significantly affect the viscosity of an aqueous composition to which it has been added. This effect of adding little to no viscosity can typically be taken advantage of in formulating a product herein. For example, an alpha-glucan ether derivative can be used instead of an incumbent ingredient that provides unweildy or undesirable effects on product viscosity.

In some aspects, a product herein can be in the form of, or comprise, a fabric care composition. A fabric care composition can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition. In other embodiments, a fabric care composition can be in a dry form such as a granular detergent or dryer-added fabric softener sheet. Other non-limiting examples of fabric care compositions can include: granular or powder-form all-purpose or heavy-duty washing agents; liquid, gel or paste-form all-purpose or heavy-duty washing agents; liquid or dry fine-fabric (e.g. delicates) detergents; cleaning auxiliaries such as bleach additives, “stain-stick”, or pre-treatments; substrate-laden products such as dry and wetted wipes, pads, or sponges; sprays and mists; water-soluble unit dose articles. As further examples, a composition herein can be in the form of a liquid, gel, powder, hydrocolloid, aqueous solution, granule, tablet, capsule, bead or pastille, single compartment sachet, multi-compartment sachet, single compartment pouch, or multi-compartment pouch.

A detergent composition herein may be in any useful form, e.g., as powders, granules, pastes, bars, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition (e.g., of a fabric care product or any other product herein) typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein can optionally comprise one or more detergent builders or builder systems. In some aspects, oxidized alpha-1,3-glucan can be included as a co-builder; oxidized alpha-1,3-glucan compounds for use herein are disclosed in U.S. Patent Appl. Publ. No. 2015/0259439. In some aspects incorporating at least one builder, the cleaning compositions comprise at least about 1%, from about 3% to about 60%, or even from about 5% to about 40%, builder by weight of the composition. Examples of builders include alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949).

In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer-inhibiting agents. Suitable polymeric dye transfer-inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer-inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer-inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo- or co-polymeric acids or their salts, in which the polycarboxylic acid comprises at least two carboxyl radicals separated from each other by not more than two carbon atoms.

A detergent composition herein may additionally comprise one or more enzymes as disclosed above, for example. In some aspects, a detergent composition can comprise one or more enzymes, each at a level from about 0.00001% to about 10% by weight of the composition and the balance of cleaning adjunct materials by weight of composition. In some other aspects, a detergent composition can also comprise each enzyme at a level of about 0.0001% to about 10%, about 0.001% to about 5%, about 0.001% to about 2%, or about 0.005% to about 0.5%, by weight of the composition. Enzymes comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in some aspects may comprise one or more other types of polymer in addition to an alpha-glucan ether derivative as disclosed herein. Examples of other types of polymers useful herein include carboxymethyl cellulose (CMC), dextran, poly(vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

Examples of suitable anti-redeposition and/or clay soil removal agents for a fabric care product herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and International Patent Appl. Publ. No. WO95/32272, all of which are incorporated herein by reference.

Particular forms of detergent compositions that can be adapted for purposes herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911 A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include at least one dye transfer-inhibiting agent, examples of which are described above.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra(methylenephosphonic acid).

A detergent herein such as that for fabric care (e.g., laundry) can be comprised in a unit dose (e.g., sachet or pouch), for example. A unit dose form can comprise a water-soluble outer film that completely envelopes a liquid or solid detergent composition. A unit dose can comprise a single compartment, or at least two, three, or more (multiple) compartments. Multiple compartments can be arranged in a superposed orientation or a side-by-side orientation. A unit dose herein is typically a closed structure of any form/shape suitable for holding and protecting its contents without allowing contents release prior to contact with water.

Products disclosed herein can be in the form of, or comprise, a fabric softener (liquid fabric softener), for example. An example of such a composition is a rinse used in laundering a fabric-comprising material herein typically following cleaning of the fabric-comprising material with a laundry detergent composition (e.g., laundry rinse such as used in a laundry rinse cycle in a washing machine). The concentration of an alpha-glucan ether herein in a composition comprising fabric softener (e.g., a rinse) can be about, or at least about, 20, 30, 40, 50, 60, 70, 80, 20-80, 20-70, 20-60, 30-80, 30-70, 30-60, 40-80, 40-70, or 40-60 ppm, for example. The concentration of a fabric softener in a composition (e.g., a rinse) can be about, or at least about, 50, 75, 100, 150, 200, 300, 400, 500, 600, 50-600, 50-500, 50-400, 50-300, 50-200, 100-600, 100-500, 100-400, 100-300, 100-200, 10-600, 50-500, 50-400, 50-300, 50-200, 200-600, 200-500, 200-400, or 200-300 ppm, for example. Fabric softener concentration can be based on the total fabric softener composition added (not necessarily based on an individual component of the fabric softener), or based on one or more fabric softening agents(s) in the fabric softener formulation. A fabric softener herein can further comprise, for example, one or more of a fabric softening agent (e.g., diethyl ester dimethyl ammonium chloride), anti-static agent, perfume, wetting agent, viscosity modifier (e.g., calcium chloride), pH buffer/buffering agent (e.g., formic acid), antimicrobial agent, anti-oxidant, radical scavenger (e.g., ammonium chloride), chelant/builder (e.g., diethylenetriamine pentaacetate), anti-foaming agent/lubricant (e.g., polydimethylsiloxane), preservative (e.g., benzisothiazolinone) and colorant. In some aspects, a fabric softener can further comprise one or more of a fabric softening agent, viscosity modifier, pH buffer/buffering agent, radical scavenger, chelant/builder and anti-foaming agent/lubricant. A fabric softener can be perfume-free and/or dye-free, or have less than about 0.1 wt % of a perfume and/or dye in some aspects. In some aspects, a fabric softener that can be adapted for use herein can be as disclosed in any of U.S. Patent Appl. Publ. Nos. 2014/0366282, 2001/0018410, 2006/0058214, 2021/0317384, or 2006/0014655, or Int. Patent Appl. Publ. Nos. WO2007/078782, WO1998/016538, WO1998/012293, WO1998007920, WO2000/070004, WO2009/146981, WO2000/70005, or WO2013087366, which are incorporated herein by reference. Some brands of fabric softeners that can be adapted for use herein, if desired, include DOWNY, DOWNY ULTRA, DOWNY INFUSIONS, ALL, SNUGGLE, LENOR and GAIN. A liquid fabric softener product (e.g., as it exists before being used in a laundry rinse cycle) can be formulated to include at least one alpha-glucan ether derivative in some aspects. A fabric softener in some aspects can be in a unit dose, such as disclosed herein for a detergent.

Products disclosed herein can be in the form of, or comprise, a dishwashing detergent composition, for example. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in some aspects of a dishwashing detergent composition include, for example, one or more of a phosphate; oxygen- or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly(oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N,N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl- and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt(III) and related complexes); (vii) a metal care agent in the range from about 0.1 wt % to 5 wt % (e.g., benzatriazoles, metal salts and complexes, and/or silicates); (viii) a glass corrosion inhibitor in the range of about 0.1 wt % to 5 wt % (e.g., a salt and/or complex of magnesium, zinc, or bismuth); and/or (ix) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts). In some aspects, a dishwashing detergent ingredient or entire composition (but adapted accordingly to comprise an alpha-glucan ether derivative herein) can be as disclosed in U.S. Pat. Nos. 8575083 or 9796951, U.S. Pat. Appl. Publ. No. 2017/0044468, or Int. Pat. Appl. Publ. No. WO2023/111170, which are each incorporated herein by reference.

A detergent herein such as that for dish care can be comprised in a unit dose (e.g., sachet or pouch) (e.g., water-soluble unit dose article), for example, and can be as described above for a fabric care detergent, but rather comprise a suitable dish detergent composition.

It is believed that numerous commercially available detergent formulations can be adapted to include an alpha-glucan ether derivative as disclosed herein. Examples of commercially available detergent formulations include PUREX® ULTRAPACKS (Henkel), FINISH® QUANTUM (Reckitt Benckiser), CLOROX™ 2 PACKS (Clorox), OXICLEAN MAX FORCE POWER PAKS (Church & Dwight), TIDE® STAIN RELEASE, CASCADE® ACTIONPACS, and TIDE® PODS™ (Procter & Gamble).

Products disclosed herein can be in the form of, or comprise, an oral care composition, for example. Examples of oral care compositions include dentifrices, toothpaste, mouth wash, mouth rinse, chewing gum, and edible strips that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ~0.1-10 wt % or ~0.1-5.0 wt %, ~0.1-2.0 wt %) of an alpha-glucan ether derivative as disclosed herein, for example. An alpha-glucan ether derivative comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which an alpha-glucan ether derivative herein can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g., azacycloalkane-2,2-diphosphonates such as azacycloheptane-2,2-diphosphonic acid), N-methyl azacyclopentane-2,3-diphosphonic acid, ethane-1-hydroxy-1, 1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

Additional examples of personal care, household care, and other products and ingredients herein can be any as disclosed in U.S. Pat. No. 8,796,196, which is incorporated herein by reference. Examples of personal care, household care, and other products and ingredients herein include perfumes, fragrances, air odor-reducing agents, insect repellents and insecticides, bubble-generating agents such as surfactants, pet deodorizers, pet insecticides, pet shampoos, disinfecting agents, hard surface (e.g., floor, tub/shower, sink, toilet bowl, door handle/panel, glass/window, car/automobile exterior or interior) treatment agents (e.g., cleaning, disinfecting, and/or coating agents), wipes and other non-woven materials, colorants, preservatives, antioxidants, emulsifiers, emollients, oils, medicaments, flavors, and suspending agents.

The present disclosure also concerns a method of treating a material with a product herein. This method comprises contacting a material with an aqueous product comprising at least one alpha-glucan ether derivative as disclosed herein.

A material contacted with an aqueous product in a contacting method herein can comprise a fabric in some aspects. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelasse, oxford, percale, poplin, plisse, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

An aqueous product that is contacted with a fabric can be, for example, a fabric care composition (e.g., laundry detergent, fabric softener). Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference. In other examples, a material comprising fabric can be contacted with an aqueous composition herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95 °C (e.g., for laundry wash or rinse: a “cold” temperature of about 15-30 °C, a “warm” temperature of about 30-50 °C, a “hot” temperature of about 50-95 °C); (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as dissolving, mixing, shaking, spraying, treating, immersing, flushing, pouring on or in, combining, painting, coating, applying, affixing to, and/or communicating an effective amount of an alpha-glucan ether derivative herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a PhabrOmeter® System for measuring relative hand value (available from Nu Cybertek, Inc. Davis, CA) (American Association of Textile Chemists and Colorists [AATCC test method “202-2012, Relative Hand Value of Textiles: Instrumental Method” ]).

In some aspects of treating a material comprising fabric, an alpha-glucan ether derivative of the aqueous product adsorbs to the fabric. This feature is believed to render an alpha-glucan ether derivative herein useful as an anti-redeposition agent and/or anti-greying agent in fabric care compositions (in addition to its viscosity-modifying effect, e.g.). An anti-redeposition agent or anti-greying agent herein helps keep soil from redepositing onto clothing in wash water after the soil has been removed. It is further contemplated that adsorption of an alpha-glucan ether derivative herein to a fabric enhances mechanical properties of the fabric in some aspects.

Adsorption of an alpha-glucan ether derivative to a fabric herein can be measured using a colorimetric technique (e.g., Dubois et al., 1956, Anal. Chem. 28:350-356; Zemljic et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, melamine, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed herein and in U.S. Pat. No. 8,575,083 and U.S. Pat. Appl. Publ. No. 2017/0044468, which are incorporated herein by reference. In some aspects, a tableware article can be contacted with an aqueous composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method also include a surface of the integumentary system such as skin, hair or nails (i.e., any keratin-comprising tissue or material) (e.g., with a body wash, skin conditioner, shampoo, hair conditioner, nail conditioner, or any other suitable product herein).

Thus, some aspects of the present disclosure concern material (e.g., fabric, or a fiber-comprising product as disclosed herein, or any other material herein such as hair, skin, or other keratin-comprising material) that comprises an alpha-glucan ether derivative herein. Such material can be produced following a material treatment method as disclosed herein, for example. A material may comprise an alpha-glucan ether derivative in some aspects if the alpha-glucan ether derivative is adsorbed to, or otherwise in contact with (e.g., alpha-glucan ether comprised in a coating of the material), the surface of the material.

Some aspects of a method of treating a material herein further comprise a drying step, in which a material is dried after being contacted with the aqueous composition. A drying step can be performed directly after the contacting step, or following one or more additional steps that might follow the contacting step (e.g., drying of fabric, tableware, or hair after being rinsed, in water for example, following a wash in an aqueous composition herein). Drying can be performed by any of several means known in the art, such as air drying (e.g., ~20-25 °C), or at a temperature of at least about 30, 40, 50, 60, 70, 80, 90, 100, 120, 140, 160, 170, 175, 180, or 200 °C, for example. A material that has been dried herein typically has less than 3, 2, 1, 0.5, or 0.1 wt % water comprised therein.

An aqueous product used in a treatment method herein can be any aqueous product/composition disclosed herein. Examples of aqueous products include detergents (e.g., laundry detergent or dish detergent), fabric softeners, water-containing dentifrices such as toothpaste, and hair care products such as hair styling, hair cleaning, or hair conditioning products.

Some aspects herein regard a method of treating hair (e.g., washing and/or conditioning). Such a method can comprise, for example, at least steps:
(a) contacting (e.g., coating) hair with an aqueous product (e.g., shampoo and/or conditioner) comprising an alpha-glucan ether derivative herein, typically wherein the aqueous product is applied in a manner in which it has been diluted in water (e.g., by virtue of using in a shower/bath setting), thereby providing treated hair (or coated hair),
(b) typically rinsing the treated hair with water (typically warm water as used in a shower/bath setting),
(c) optionally drying the treated hair following step (a) or step (b).

Step (c) of drying can be performed following any suitable drying process herein, for example, such as by air drying or blow drying, with either room temperature or heated air. Drying can be done with (or without) agitation of the treated hair, such as by combing or brushing while drying.

Wet treated hair (e.g., rinsed treated hair) and/or dry treated hair resulting from such a hair treatment method can require a lower amount of energy (e.g., lessened by about, or at least about, 5%, 6%, 7%, 8%, 9%, 10%, 11%, 12%, 13%, 14%, 15%, 16%, 17%, 18%, 19%, 20%) to be combed (e.g., as compared to hair otherwise treated in the same manner but with a hair care composition that does not comprise an alpha-glucan ether derivative herein and that optionally instead comprises an incumbent/conventional hair treatment/conditioning polymer such as polyquaternium-10, guar hydroxypropyltrimonium chloride, polyquaternium-7, hydroxypropyl guar hydroxypropyltrimonium chloride). Hair combing energy can be measured as disclosed in U.S. Pat. Appl. Publ. No. 2014/0271504 (incorporated herein by reference) or as described in the below Examples, for example. In some aspects, a method of treating hair further comprises step (d) of combing or brushing the treated hair, where such combing/brushing requires less combing energy as described above.

Hair treated herein can be of any hair type, such as straight, wavy, curly, or coily (kinky) hair. Straight hair can be fine to coarse, and/or curl-resistant, for example. Wavy hair can be fine and thin, to coarse and frizzy, for example. Curly hair can be of loose curls to corkscrew curls, for example. Coily hair can be tightly coiled to Z-angled coiled, for example. Hair in some aspects before being treated with an alpha-glucan ether derivative herein can be virgin hair, which has never been dyed, bleached, or chemically processed (e.g., a perm), for example, or it can be non-virgin hair (e.g., dyed, beached and/or chemically treated). Hair herein to which an alpha-glucan ether derivative has been adsorbed or otherwise deposited on the hair surface can be of any of the foregoing hair types, for instance.

Non-limiting examples of compositions and methods disclosed herein include:

1. A composition (typically can be characterized as a liquid composition) comprising: (i) about 15% to 75% (e.g., about 20% to 70%) by weight of at least one organic solvent (typically a polar organic solvent), (ii) about 20% to 50% by weight of at least one cationic alpha-glucan ether derivative, and (iii) less than about 50% (e.g., less than about 45%) by weight water (but typically also at least about 20% by weight water); wherein at least about 50% of the glycosidic linkages of the cationic alpha-glucan ether derivative are alpha-1,6 linkages (i.e., the ether is a cationic alpha-1,6-glucan ether, or cationic dextran ether), and the cationic alpha-glucan ether derivative has a degree of substitution (DoS) of about 0.001 to about 3.0 with at least one positively charged organic group that is ether-linked to the alpha-glucan.

2. The composition of embodiment 1, wherein at least about 90% of the glycosidic linkages of the cationic alpha-glucan ether derivative are alpha-1,6 linkages.

3. The composition of embodiment 1 or 2, wherein the cationic alpha-glucan ether derivative comprises at least about 1% alpha-1,2 and/or alpha-1,3 branches (e.g., about, or at least about, 3%, 3-35%, 3-30%, 3-25%, or 3-20% alpha-1,2 and/or alpha-1,3 branches).

4. The composition of embodiment 1, 2, or 3, wherein: (a) the alpha-glucan of the cationic ether derivative has a weight-average molecular weight (Mw) of about 0.9 kDa to 450 kDa (e.g., about 10-350, 50-350, 90-300, 125-250, 150-250, 150-200, or 175-200 kDa), or (b) the cationic alpha-glucan ether derivative has an Mw of about 1 kDa to 500 kDa (e.g., 10-400, 40-300, 80-300, 100-250, 150-250, 180-225, or 180-200 kDa).

5. The composition of embodiment 1, 2, 3, or 4, wherein the DoS is about 0.01 to 1.5 (e.g., about 0.01-1.0, 0.01-0.8, 0.03-0.7, 0.04-0.6, or 0.05-0.5).

6. The composition of embodiment 1, 2, 3, 4, or 5, wherein the positively charged organic group comprises a substituted ammonium group.

7. The composition of embodiment 6, wherein the substituted ammonium group comprises a quaternary ammonium group.

8. The composition of embodiment 7, wherein the quaternary ammonium group comprises a trimethylammonium group.

9. The composition of embodiment 7, wherein the quaternary ammonium group comprises at least one C₁₀ to C₁₆ alkyl group (e.g., comprises a C₁₀ to C₁₆ alkyl group and two C₁ to C₄ alkyl groups).

10. The composition of embodiment 1, 2, 3, 4, 5, 6, 7, 8, or 9, wherein the positively charged organic group comprises a quaternary ammonium hydroxyalkyl group (e.g., a quaternary ammonium hydroxymethyl group, a quaternary ammonium hydroxyethyl group, or a quaternary ammonium hydroxypropyl group).

11. The composition of embodiment 10, wherein the quaternary ammonium hydroxyalkyl group comprises a trimethylammonium hydroxyalkyl group (e.g., a trimethylammonium hydroxymethyl group, a trimethylammonium hydroxyethyl group, or a trimethylammonium hydroxypropyl group).

12. The composition of embodiment 1, 2, 3, 4, 5, 6, 7, 8, 9, 10, or 11, wherein the at least one organic solvent comprises ethanol, ethylene glycol, polyethylene glycol, 1,2-propanediol, propylene glycol, dipropylene glycol, tripropyleneglycol, polypropylene glycol, and/or glycerol.

12b. The composition of embodiment 12, wherein the at least one organic solvent comprises propylene glycol.

13. The composition of embodiment 1, 2, 3, 4, 5, 6, 7, 8, 9, 10, 11, 12, or 12b, comprising: (i) about 25% to 40% (e.g., 25-35%, 30-40%, 30-35%, 32-35%, 32-34%, 33-34%) by weight of the at least one organic solvent, (ii) about 25% to 40% (e.g., 25-35%, 30-40%, 30-35%, 32-25%, 32-34%, 33-34%) by weight of the at least one cationic alpha-glucan ether derivative, and (iii) about 25% to 40% (e.g., 25-35%, 30-40%, 30-35%, 32-35%, 32-34%, 33-34%) by weight water.

14. A product comprising the composition of embodiment 1, 2, 3, 4, 5, 6, 7, 8, 9, 10, 11, 12, 12b, or 13, or a product comprising the composition as produced by the method of embodiment 23, 24 or 25, typically wherein the composition was used as an ingredient/component in producing the product.

15. The product of embodiment 14, wherein the product is a household care product, personal care product, industrial product, or pharmaceutical product.

16. The product of embodiment 14 or 15, wherein the product is (i) a hair shampoo or hair conditioner (or any hair treatment product that typically is intended to be applied to hair and rinsed off), or (ii) a skin cleanser, soap, or other skin-washing product (or any skin treatment product that typically is intended to be applied to skin and rinsed off, typically but optionally for purposes of cleaning or cleansing the skin).

17. The product of embodiment 14, 15, or 16, wherein the product is, or comprises, an aqueous composition.

18. The product of embodiment 14, 15, 16, or 17, further comprising at least one surfactant.

19. The product of embodiment 14, 15, 16, 17, or 18, further comprising at least one enzyme.

20. The product of embodiment 19, wherein the enzyme is a cellulase, protease, amylase, or nuclease.

21. The product of embodiment 14, 15, 16, 17, 18, 19, or 20, further comprising at least one of a complexing agent, soil release polymer, surfactancy-boosting polymer, bleaching agent, bleach activator, bleaching catalyst, fabric conditioner, clay, foam booster, suds suppressor, anti-corrosion agent, soil-suspending agent, anti-soil re-deposition agent, dye, bactericide, tarnish inhibitor, optical brightener, perfume, saturated or unsaturated fatty acid, dye transfer-inhibiting agent, chelating agent, hueing dye, visual signaling ingredient, anti-foam, structurant, thickener, anti-caking agent, starch, sand, or gelling agent.

22. The product of embodiment 14, 15, 16, 17, 18, 19, 20, or 21, wherein the product is in the form of, or comprised in, a liquid, gel, powder, hydrocolloid, granule, tablet, bead or pastille, single-compartment sachet, multi-compartment sachet, single-compartment pouch, or multi-compartment pouch.

23. A method (process) of producing a composition (liquid composition) according to embodiment 1, 2, 3, 4, 5, 6, 7, 8, 9, 10, 11, 12, 12b, or 13, the method comprising: (a) providing an aqueous composition comprising the cationic alpha-glucan ether derivative, (b) mixing, into the aqueous composition, the organic solvent (a suitable amount thereof to achieve a desired concentration of the organic solvent in the final composition), and (c) optionally concentrating (removing water, such as by evaporation) the cationic alpha-glucan ether derivative and the organic solvent in the aqueous composition following step (b) (e.g., if necessary to reach particular concentration[s] of the cationic alpha-glucan ether derivative and/or organic solvent).

24. The method of embodiment 23, wherein the aqueous composition provided in step (a) is an etherification reaction composition (typically a terminated/quenched/neutralized reaction) in which the cationic alpha-glucan ether derivative was produced.

25. The method of embodiment 24, wherein step (a) comprises subjecting the etherification reaction composition to one or more rounds of a purification process (to increase the purity of the cationic alpha-glucan ether derivative in the aqueous composition) (e.g., diafiltration such as ultrafiltration or nanofiltration, or dialysis).

26. The product of embodiment 14, 15, 16, 17, 18, 19, 20, 21, or 22, wherein the product comprises the at least one cationic alpha-glucan ether derivative, but does not necessarily comprise the at least one organic solvent and/or the water, or does not necessarily comprise the organic solvent and/or water (or the cationic alpha-glucan ether derivative) in the stated weight percentages (e.g., the product was not made using the composition of embodiment 1 as an ingredient for making the product).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

Representative Preparation of Alpha-1,6-Glucan with Alpha-1,2 Branching

Methods to prepare alpha-1,6-glucan containing various amounts of alpha-1,2 branching are disclosed in U.S. Appl. Publ. No. 2018/0282385, which is incorporated herein by reference. Reaction parameters such as sucrose concentration, temperature, and pH can be adjusted to provide alpha-1,6-glucan having various levels of alpha-1,2-branching and molecular weight. A representative procedure for the preparation of alpha-1,2-branched alpha-1,6-glucan is provided below (containing 19% alpha-1,2-branching and 81% alpha-1,6 linkages). The 1D ¹H-NMR spectrum was used to quantify glycosidic linkage distribution. Additional samples of alpha-1,6-glucan with alpha-1,2-branching were prepared similarly. For example, one sample contained 32% alpha-1,2-branching and 68% alpha-1,6 linkages, and another contained 10% alpha-1,2-branching and 90% alpha-1,6 linkages.

Soluble alpha-1,6-glucan with about 19% alpha-1,2 branching was prepared using stepwise combination of glucosyltransferase (dextransucrase) GTF8117 and alpha-1,2 branching enzyme GTFJ18T1, according to the following procedure. A reaction mixture (2 L) comprised of sucrose (450 g/L), GTF8117 (9.4 U/mL), and 50 mM sodium acetate was adjusted to pH 5.5 and stirred at 47 °C. Aliquots (0.2-1 mL) were withdrawn at predetermined times and quenched by heating at 90 °C for 15 minutes. The resulting heat-treated aliquots were passed through a 0.45-μm filter. The flow-through was analyzed by HPLC to determine the concentration of sucrose, glucose, fructose, leucrose, oligosaccharides and polysaccharides. After 23.5 hours, the reaction mixture was heated to 90 °C for 30 minutes. An aliquot of the heat-treated reaction mixture was passed through a 0.45-μm filter and the flow-through was analyzed for soluble mono/disaccharides, oligosaccharides, and polysaccharides. A major product was linear dextran with a DPw of 93.

A second reaction mixture was prepared by adding 238.2 g of sucrose and 210 mL of alpha-1,2-branching enzyme GTFJ18T1 (5.0 U/mL) to the leftover heat-treated reaction mixture that was obtained from the GTF8117 reaction described immediately above. The mixture was stirred at 30 °C with a volume of ~2.2 L. Aliquots (0.2-1 mL) were withdrawn at predetermined times and quenched by heating at 90 °C for 15 minutes. The resulting heat-treated aliquots were passed through a 0.45-μm filter. The flow-through was analyzed by HPLC to determine the concentration of sucrose, glucose, fructose, leucrose, oligosaccharides and polysaccharides. After 95 hours, the reaction mixture was heated to 90 °C for 30 minutes. An aliquot of the heat-treated reaction mixture was passed through a 0.45-μm filter and the flow-through was analyzed for soluble mono/disaccharides, oligosaccharides, and polysaccharides. Leftover heat-treated mixture was centrifuged using 1-L centrifugation bottles. The supernatant was collected and cleaned more than 200-fold using an ultrafiltration system with 1- or 5-kDa MWCO cassettes and deionized water. The cleaned oligo/polysaccharide product solution was dried. Dry sample was then analyzed by ¹H-NMR spectroscopy to determine the anomeric linkages of the oligosaccharides and polysaccharides.

Various water-soluble alpha-1,2-branched alpha-1,6-glucans can be made following the above (or similar) enzymatic reaction strategy, for example. This type of alpha-glucan material can also be produced according to methodology disclosed in U.S. Pat. Appl. Publ. No. 2018/0282385, for example, which is incorporated herein by reference. Examples of different alpha-1,2-branched alpha-1,6-glucans that have been produced are listed in Table 1. In each of these alpha-glucans, the alpha-1,6-glucan backbone (from which there are alpha-1,2 branches) has 100% alpha-1,6 glycosidic linkages, the listed molecular weight is that of the alpha-1,6-glucan backbone. Each alpha-1,2-branch consists of a single (pendant) glucose unit.

TABLE 1
Alpha-1,2-Branched Alpha-1,6-Glucan

| Backbone | Percent Alpha-1,2 |
|---|---|
| MW (kDa) | Branching |
| 17 | 40% |
| 17 | 45% |
| 40 | 20% |
| 40 | 40% |
| 60 | 20% |
| 109 | 26% |
| 185 | 5% |
| 185 | 20% |
| 194 | 41% |
| 200 | 10% |
| 200 | 20% |
| 300 | 45% |

### Example 1

### Preparation of Cationic Alpha-Glucan Ether Formulations

This Example describes reaction and processing steps for producing a cationic alpha-glucan ether compound and formulations comprising this compound in a liquid organic medium. In particular, trimethylammonium hydroxypropyl alpha-1,2-branched alpha-1,6-glucan ether was prepared in formulations further comprising water and propylene glycol. Such liquid formulations can be used as an ingredient in producing various products, such as those disclosed herein.

Any alpha-1,2-branched alpha-1,6-glucan as disclosed herein (e.g., Table 1) can be used as a substrate for these etherification and processing procedures, for example. Examples of trimethylammonium hydroxypropyl alpha-1,2-branched alpha-1,6-glucan ether products that can be produced include those listed in Table 2.

TABLE 2
Trimethylammonium Hydroxypropyl Alpha-
1,2-Branched Alpha-1,6-Glucan Ethers

Alpha-Glucan

| | Alpha-1,2 | Ether |
|---|---|---|
| MW (kDa) | Branching | DoS |
| 40 | 40% | 0.5 |
| 40 (75)** | 40% | 0.5 |
| 17 | 40% | 0.3 |
| 40 (84)** | 40% | 0.8 |
| 109 (148)** | 26% | 0.51 |
| 194 (245)** | 41% | 0.50 |
| 194 (269)** | 41% | 0.7 |
| 185 | 5% | 0.15 |
| 185 | 5% | 0.38 |
| 185 | 5% | 0.03 |
| 200 | 20% | 0.21 |
| 200 | 10% | 0.19 |
| 185 | 5% | 0.05 |
| 185 | 20% | 0.40 |
| 185 | 5% | 0.07 |
| 185 | 5% | 0.11 |
| 185 | 5% | 0.59 |
| 109 | 26% | 0.22 |

**Parenthetical number is molecular weight of ether compound (i.e., alpha-glucan plus derivatized cationic ether groups).
Abbreviations: MW, molecular weight. DoS, degree of substitution.

Three separate reactions were performed, respectively, in 100-L, 100-L and 500-L jacketed stainless steel reactors, each equipped with a pitched blade turbine (PBT) impeller and mixer. Temperature was maintained using a recirculating water bath connected to the reactor jacket. Reaction volumes were at 90 L, 90 L and 450 L, respectively, and charged with 372 g/L of a water-soluble alpha-1,2-branched alpha-1,6-glucan. An external recirculation loop was used to charge 7.65 g/L of sodium hydroxide into each reactor. Once the temperature of each preparation stabilized at 50 °C, a recirculation loop was used to charge 2,3-epoxypropyltrimethylammonium chloride (EPTAC, to 48.7 g/L) into each reactor. Conditions were maintained for 5 hours, after which the reactions were neutralized to pH 5-7 using 10 wt % sulfuric acid, thereby terminating the reactions.

Each neutralized reaction was entered into ultrafiltration (UF) purification with 5-kDa cut-off polyethersulfone (PES) membranes and three diafiltration washes. Afterwards, another UF was run on each sample to render an alpha-glucan ether polymer concentrate of 15-18 wt % solids. The concentration was measured using refractive index (RI) and approximately 50 g of each polymer concentrate was set aside for DoS analysis by NMR and total Kjeldahl nitrogen determination. Derivatization impurities analysis of residual EPTAC, 3-chloro-2-hydroxypropyltrimethylammonium chloride (CHPTAC) and (2,3-dihydroxypropyl)trimethylammonium chloride (DHPTAC) was determined by ion chromatography (IC) analysis. Propylene glycol was mixed into each polymer concentrate at equal solids concentration (15-18 wt %). Potassium sorbate preservative was added as a preservative.

Each ether polymer mixture was then placed into an evaporator at 50-60 °C to remove ~33.2 wt % water. Approximately 50 g of each mixture was sampled to determine the concentrations of alpha-glucan ether polymer and propylene glycol using RI analysis and vacuum oven solids concentrations. The final composition of each formulation was ~33.3 wt % trimethylammonium hydroxypropyl alpha-1,2-branched alpha-1,6-glucan ether, ~33.3 wt % propylene glycol, ~33.3 wt % water, and potassium sorbate (Table 3A). The viscosity of each formulation was measured at 35 °C using an Anton Paar RheolabQC with CC27 spindle (Table 3A).

TABLE 3A
Cationic Alpha-Glucan Ether Formulations*

| | | Sample 1 | Sample 2 | Sample 3 |
|---|---|---|---|---|
| Total Nitrogen | g/100 g | 0.194 | 0.202 | 0.166 |
| (Kjeldahl) | | | | |
| Cationic Alpha- | wt % | 35.2% | 35.4% | 34.9% |
| Glucan | | | | |
| Propylene Glycol | wt % | 30.7% | 31.0% | 32.2% |
| Viscosity | cP | 9799 | 9561 | 10571 |
| (at 10^{-s}, 35 °C) | | | | |
| EPTAC | mg/L | 10.6 | 10.9 | 11.0 |
| CHPTAC | mg/L | 18.4 | 15.4 | 18.3 |
| DHPTAC | mg/L | 208 | 140 | 317 |

*Most of the contents balance of each sample was water.

Table 3B provides additional cationic alpha-glucan ether formulations that were produced generally following the above methodology.

TABLE 3B
Cationic Alpha-Glucan Ether Formulations

| | | Sample 4 | Sample 5 | Sample 6 | Sample 7 |
|---|---|---|---|---|---|
| Cationic Alpha- | wt % | 30.0% | 30.0% | 30.0% | 33.3% |
| Glucan | | | | | |
| Propylene Glycol | wt % | 36.0% | 38.5% | 40.0% | 33.3% |
| Water | wt % | 34.0% | 31.5% | 30.0% | 33.3% |

Analytical Results

| | | | | | |
|---|---|---|---|---|---|
| Viscosity | cP | 15562 | 18729 | 18835 | 25499 |
| (at 10^{-s}, 20 °C) | | | | | |
| Water activity | a_{w} | 0.774 | 0.753 | 0.745 | 0.815 |

The liquid compositions in this Example and of the present disclosure have improved processibility and reduced water content. An organic solvent component (e.g., propylene glycol) of the liquid formulations enabled improved processibility while also reducing water content. It was important to control the water content to ensure that a liquid formulation would not negatively affect the packaging integrity of a single unit dose product containing the liquid formulation as an ingredient.

### Example 2

Effect on Hair Combability from Using Shampoo Formulations Containing Cationic Alpha-1,6-Glucan Ether

This Example describes whether, and to what extent, including a cationic alpha-glucan ether (trimethylammonium hydroxypropyl alpha-1,2-branched alpha-1,6-glucan ether) in shampoo formulations affects the ability of such formulations to render shampoo-washed hair to be more combable. Hair combability is one of the attributes immediately identified by consumers during and after using shampoo. Hair combability relates to hair fiber conditioning by a shampoo or other hair treatment: the more a hair fiber is rendered smooth by a conditioning agent, the less energy is needed to comb the conditioned hair (i.e., decreased combing energy).

### Protocol

### Pre-Treatment:

Fifty tresses were prepared from natural Caucasian hair weighing 2.5 g each and 25 cm long. All tresses underwent a standard pre-cleaning process with 10% sodium lauryl ether sulfate (SLES) solution for 1 minute and then rinsed with running water.

### Base Line Combability Measurement (Wet and Dry):

Wet combability was first measured (Wet Baseline). Tresses were then dried in a controlled environment at 55±5% relative humidity and 22±2 °C for 24 hours before dry-combing tests. The dry combability of each tress was then measured (Dry Baseline). The fifty tresses were split to ten groups and submitted to various shampoo treatments (below).

### Final Combability Measurement (Wet and Dry):

Measurements of final wet combability were taken of each tress following shampoo washing (Wet Final). Then, the hair tresses were dried in a controlled environment at 55±5% relative humidity and 22±2 °C for 24 hours before dry-combing tests. The dry combability of each tress was then measured (Dry Final).

### Formula and Test Results: Shampoo-1

A trimethylammonium hydroxypropyl alpha-1,2-branched alpha-1,6-glucan ether (cationic alpha-1,6-glucan ether herein below) and four conventional hair conditioning polymers (polyquaternium-10, guar hydroxypropyltrimonium chloride, polyquaternium-7, hydroxypropyl guar hydroxypropyltrimonium chloride) were individually used in a typical shampoo formulation comprising sodium laureth sulfate and cocamidopropyl betaine (Table 4) as surfactants. The cationic alpha-1,6-glucan ether was entered into formulations 1B and 1C using a composition comprising about 33 wt % water, 33 wt % propylene glycol, and 33 wt % of the ether.

TABLE 4
Components of Shampoo-1 Formulations 1A-1G

| | 1A | 1B | 1C | 1D | 1E | 1F | 1G |
|---|---|---|---|---|---|---|---|
| Cationic Alpha-1,6- | 0.00 | 0.20 | 0.40 | 0.00 | 0.00 | 0.00 | 0.00 |
| Glucan Ether | | | | | | | |
| Polyquaternium-10 | 0.00 | 0.00 | 0.00 | 0.20 | 0.00 | 0.00 | 0.00 |
| Guar | | | | | | | |
| Hydroxypropyltrimonium | 0.00 | 0.00 | 0.00 | 0.00 | 0.20 | 0.00 | 0.00 |
| Chloride | | | | | | | |
| Polyquaternium-7 | 0.00 | 0.00 | 0.00 | 0.00 | 0.00 | 0.20 | 0.00 |
| Hydroxypropyl Guar | 0.00 | 0.00 | 0.00 | 0.00 | 0.00 | 0.00 | 0.20 |
| Hydroxypropyltrimonium | | | | | | | |
| Chloride | | | | | | | |
| Cocamidopropyl | 2.00 | 2.00 | 2.00 | 2.00 | 2.00 | 2.00 | 2.00 |
| Betaine | | | | | | | |
| Sodium Laureth Sulfate | 12.00 | 12.00 | 12.00 | 12.00 | 12.00 | 12.00 | 12.00 |
| Phenoxyethanol | 0.80 | 0.80 | 0.80 | 0.80 | 0.80 | 0.80 | 0.80 |
| Sodium Chloride | 1.50 | 1.50 | 1.50 | 1.50 | 1.50 | 1.50 | 1.50 |
| Citric Acid Anhydrous | 0.030 | 0.031 | 0.031 | 0.031 | 0.032 | 0.030 | 0.029 |
| Disodium EDTA | 0.10 | 0.10 | 0.10 | 0.10 | 0.10 | 0.10 | 0.10 |
| Water | QS | QS | QS | QS | QS | QS | QS |

Notes:
All listed values are wt % active matter. Water added to 100 wt % total.

As seen in Table 5, cationic alpha-1,6-glucan ether-containing shampoos (formulations 1B and 1C) decreased combing energy with wet hair to a greater extent as compared to the control shampoo (formula 1A, no conditioning polymer), which means that the ether improved wet hair combability rendered by the shampoo. Also, formulations 1B and 1C outperformed all shampoos, including those containing conventional conditioning polymers (formulations 1D-1G), in affecting dry combability (Table 5).

TABLE 5
Percent Reduction of Combing Energy When
Using Shampoo-1 Formulations 1A-1G (from
baseline, before sample treatment)

| | 1A | 1B | 1C | 1D | 1E | 1F | 1G |
|---|---|---|---|---|---|---|---|
| Wet combability | 53% | 57% | 69% | 63% | 65% | 60% | 65% |
| Dry combability | 69% | 73% | 74% | 68% | 67% | 64% | 50% |

### Formula and Test Results: Shampoo-2

The cationic alpha-1,6-glucan ether and four conventional hair conditioning polymers used above in Shampoo-1 were individually used in a typical non-sulfate shampoo formulation comprising sodium C14-C16 olefin sulfonate (a commonly used non-sulfate anion) and cocamidopropyl betaine as surfactants (Table 6). The cationic alpha-1,6-glucan ether was entered into formulations 2B and 20 using a composition comprising about 33 wt % water, 33 wt % propylene glycol, and 33 wt % of the ether.

TABLE 6
Components of Shampoo-2 Formulations 2A-2G

| | 2A | 2B | 2C | 2D | 2E | 2F | 2G |
|---|---|---|---|---|---|---|---|
| Cationic Alpha-1,6- | 0.00 | 0.20 | 0.40 | 0.00 | 0.00 | 0.00 | 0.00 |
| Glucan Ether | | | | | | | |
| Polyquaternium-10 | 0.00 | 0.00 | 0.00 | 0.20 | 0.00 | 0.00 | 0.00 |
| Guar | 0.00 | 0.00 | 0.00 | 0.00 | 0.20 | 0.00 | 0.00 |
| Hydroxypropyltrimonium | | | | | | | |
| Chloride | | | | | | | |
| Polyquaternium-7 | 0.00 | 0.00 | 0.00 | 0.00 | 0.00 | 0.20 | 0.00 |
| Hydroxypropyl Guar | 0.00 | 0.00 | 0.00 | 0.00 | 0.00 | 0.00 | 0.20 |
| Hydroxypropyltrimonium | | | | | | | |
| Chloride | | | | | | | |
| Cocamidopropyl | 2.00 | 2.00 | 2.00 | 2.00 | 2.00 | 2.00 | 2.00 |
| Betaine | | | | | | | |
| Sodium C14-C16 Olefin | 12.00 | 12.00 | 12.00 | 12.00 | 12.00 | 12.00 | 12.00 |
| Sulfonate | | | | | | | |
| Phenoxyethanol | 0.80 | 0.80 | 0.80 | 0.80 | 0.80 | 0.80 | 0.80 |
| Sodium Chloride | 3.50 | 3.50 | 3.50 | 3.50 | 3.50 | 3.50 | 3.50 |
| Citric Acid Anhydrous | 0.012 | 0.012 | 0.008 | 0.010 | 0.007 | 0.080 | 0.007 |
| Disodium EDTA | 0.10 | 0.10 | 0.10 | 0.10 | 0.10 | 0.10 | 0.10 |
| Water | QS | QS | QS | QS | QS | QS | QS |

Notes:
All listed values are wt % active matter. Water added to 100 wt % total.

As seen in Table 7, cationic alpha-1,6-glucan ether-containing shampoos (formulations 2B and 2C) decreased combing energy with dry hair to a greater extent as compared to the control shampoo (formula 2A, no conditioning polymer) and all the shampoos containing conventional conditioning polymers (formulations 2D-2G), which means that the ether improved hair combability rendered by the shampoo.

TABLE 7
Percent Reduction of Combing Energy When
Using Shampoo-2 Formulations 2A-2G (from
baseline, before sample treatment)

| | 2A | 2B | 2C | 2D | 2E | 2F | 2G |
|---|---|---|---|---|---|---|---|
| Wet combability | 27% | 13% | 16% | 7% | 25% | 5% | 16% |
| Dry combability | 61% | 65% | 72% | 48% | 58% | 58% | 57% |

### Formula and Test Results: Shampoo-3

The cationic alpha-1,6-glucan ether used above in Shampoos-1 and -2 was tested in shampoo formulations lacking silicone and compared to a shampoo formulation containing silicone (dimethicone) as a conditioning agent (Table 8). The cationic alpha-1,6-glucan ether was entered into formulations 3B and 3C using a composition comprising about 33 wt % water, 33 wt % propylene glycol, and 33 wt % of the ether.

TABLE 8
Components of Shampoo-3 Formulations 3A-3D

| | 3A | 3B | 3C | 3D |
|---|---|---|---|---|
| Cationic Alpha-1,6-Glucan | 0.00 | 0.20 | 0.40 | 0.00 |
| Ether | | | | |
| Guar | 0.20 | 0.00 | 0.00 | 0.20 |
| Hydroxypropyltrimonium | | | | |
| Chloride | | | | |
| Cocamidopropyl Betaine | 2.00 | 2.00 | 2.00 | 2.00 |
| Sodium Laureth Sulfate | 12.00 | 12.00 | 12.00 | 12.00 |
| Dimethicone | 1.00 | 0.00 | 0.00 | 0.00 |
| Phenoxyethanol | 0.80 | 0.80 | 0.80 | 0.80 |
| Sodium Chloride | 1.50 | 1.50 | 1.50 | 1.50 |
| Citric Acid Anhydrous | 0.030 | 0.031 | 0.031 | 0.032 |
| Disodium EDTA | 0.10 | 0.10 | 0.10 | 0.10 |
| Water | QS | QS | QS | QS |

Notes:
All listed values are wt % active matter. Water added to 100 wt % total.

As seen in Table 9, cationic alpha-1,6-glucan ether-containing shampoos (formulations 3B and 3C) showed similar combability effects on dry hair as with a typical shampoo having dimethicone and guar hydroxypropyltrimonium chloride (formulation 3A), but a better effect than exhibited by a silicone-free shampoo (formulation 3D). This result indicates that cationic alpha-1,6-glucan ether is a more promising option than a conventional conditioning polymer as replacement for silicone.

TABLE 9
Percent Reduction of Combing Energy When
Using Shampoo-3 Formulations 3A-3D (from
baseline, before sample treatment)

| | 3A | 3B | 3C | 3D |
|---|---|---|---|---|
| Wet combability | 33% | 57% | 69% | 65% |
| Dry combability | 73% | 73% | 74% | 67% |

TABLE 10
Reference List of Some Ingredients Used in this Example

| | |
|---|---|
| Polyquaternium-10 | Sensomer 10M Polymer |
| Guar hydroxypropyltrimonium chloride | Jaguar C14S |
| Polyquaternium-7 | Merquat 550PR Polymer |
| Hydroxypropyl Guar | Jaguar C162 |
| Hydroxypropyltrimonium Chloride | |
| Cocamidopropyl Betaine | Dehyton K COS |
| Sodium Laureth Sulfate | Texapon NSO UP |
| Sodium C14-16 Olefin Sulfonate | Nansa LSS38/AV |
| Dimethicone | Xiameter MEM-2664 |
| Phenoxyethanol | Neolone PH100 |

It was further shown that the cationic alpha-glucan ether of this Example can deposit onto hair (data not shown). This deposition capability is contemplated to account for, at least in part, the foregoing beneficial effects of using shampoo comprising the alpha-glucan ether derivative. It is contemplated that this deposition capability likewise applies to other keratin-containing tissues such as skin and nails, for example. It was also observed that the cationic alpha-glucan ether did not increase the viscosity of the above formulations, and hence, for example, is useful for formulating products that can be converted to a foam prior to use (e.g., via a foaming pump). Finally, the cationic alpha-glucan ether did not require any special steps to enable its hydration.

### Example 3

Preparation of an Additional Cationic Alpha-Glucan Ether Formulation Three grams of cationically modified alpha-1,6-glucan (alpha-1,2-branched) ether compound (in powder form, 100% active) was mixed with a total of 7 g solvent (3.5 g water and 3.5 g propylene glycol) in a glass vial and mixed with a spatula. Complete dissolution of the alpha-1,6-glucan ether compound in the solvent occurred, thereby providing 10 g of a premix (PP4). Rheology measurements were taken with this premix (Table 11).

TABLE 11

| Premix | PP4 |
|---|---|
| Cationic alpha-1,6-glucan ether compound (g) | 3 |
| Water (g) | 3.5 |
| Propylene glycol (g) | 3.5 |
| Viscosity (20 °C) | 9580 (@0.05 1/s) |
| | 2780 (@1000 1/s) |

### Example 4

### Preparation of Additional Cationic Alpha-Glucan Ether Formulations

Formulations (premixes) from Examples 1 and 3 were used as intermediate premixes to create premixes PP5, PP6 and PP7 (Table 12). Specifically, PP5 was made by mixing 60 wt % of Sample 1 (Ex. 1, Table 3A) (“PP1” below) with 40% structuring agent; PP6 was made by mixing 75 wt % of Sample 2 (Ex. 1, Table 3A) (“PP2” below) with 25% structuring agent; and PP7 was made by mixing 80 wt % of PP4 (Ex. 3, Table 11) with 20% structuring agent.

TABLE 12

| Sample | ID | PP5 | PP6 | PP7 |
|---|---|---|---|---|
| Intermediate premix | | PP1 | PP2 | PP4 |
| Intermediate premix | wt % | 60 | 75 | 80 |
| Structuring agent | wt % | 40 | 25 | 20 |
| (contain ~70% of water [w/w]) | | | | |

### Example 5

### Procedure of Making a Water-Soluble Unit Dose Article Using a Cationic Alpha-Glucan Ether Formulation

The following liquid detergent base was produced through standard mixing of the components described in Table 13:

TABLE 13

| Material | Wt % |
|---|---|
| Citric acid | 0.87 |
| 1,2-propanediol | 9.3 |
| Dipropyleneglycol | 4.63 |
| Monoethanolamine | 9.38 |
| Glycerol | 4.67 |
| HEDP chelant | 2.79 |
| K₂SO₃ | 0.06 |
| NEODOL 24/7 | 5.52 |
| HLAS | 26.69 |
| Brightener 49 | 0.02 |
| TPK fatty acid | 2.01 |
| C₁₂₁₄AE₃S | 18.11 |
| Ethoxylated polyethyleneimine (PEI₆₀₀EO₂₀) | 4.94 |
| MgCl₂ | 1.27 |
| Water | Balance |

3.8 parts of the cationic alpha-glucan ether premix, PP6 (Ex. 4, Table 12), was mixed with 96.2 parts of the liquid detergent base mentioned above (Table 13) to obtain a liquid detergent composition comprising about 1% by weight of the cationic alpha-glucan ether compound for making a soluble unit dose article (below).

A water-soluble unit dose article was made by following process steps comprising:
a. deforming a first water-soluble film to create an open cavity;
b. filling the open cavity with a liquid detergent composition comprising cationically modified alpha-1,6-glucan ether compound;
c. closing the open cavity with a water-soluble lid, wherein the water-soluble lid comprises a second water-soluble film;
d. sealing the first water-soluble film and the water-soluble lid together to create the water-soluble unit dose article.

### Example 6

Effects on Rinsability and Skin-Feel from Using Skin Cleansing/Washing Formulations Containing Cationic Alpha-1,6-Glucan Ether

This Example describes whether, and to what extent, including cationic alpha-glucan ether (trimethylammonium hydroxypropyl alpha-1,2-branched alpha-1,6-glucan ether) in skin cleansing/washing formulations affects the ability of such formulations to be rinsed from skin. The alpha-glucan ether derivative used in this Example was the same as the alpha-glucan ether derivative used in Example 2.

An evaluation (quantitative descriptive analysis) by a trained sensory panel was conducted to investigate the sensorial benefits of using skin cleansing products formulated with, or without, the cationic alpha-glucan ether. These formulations are listed in Tables 14 and 15.

TABLE 14
Components of Skin Cleansing Formulations 1A and 1B

| | 1A | 1B |
|---|---|---|
| Cationic Alpha-1,6-Glucan Ether | 0.0 | 0.4 |
| Cocamidopropyl Betaine | 3.0 | 3.0 |
| Caprylyl/Capryl Glucoside | 5.2 | 5.2 |
| Sodium Benzoate | 0.5 | 0.5 |
| Citric Acid | 0.2 | 0.2 |
| Water | QS | QS |

Notes:
All listed values are wt % active matter. Water added to 100 wt % total.

TABLE 15
Components of Skin Cleansing Formulations 2A and 2B

| | 2A | 2B |
|---|---|---|
| Cationic Alpha-1,6-Glucan Ether | 0.0 | 0.4 |
| Cocamidopropyl Betaine | 2.1 | 2.1 |
| Sodium Laureth Sulfate | 9.0 | 9.0 |
| Potassium Laurate | 3.0 | 3.0 |
| Phenoxyethanol | 0.5 | 0.5 |
| Sodium Chloride | 2.0 | 2.0 |
| Water | QS | QS |

Notes:
All listed values are wt % active matter. Water added to 100 wt % total.

The panel observed that the rinsability of Formula 1B was statistically higher than the rinsability of Formula 1A (95% confidence interval). Better rinsability can be useful for rendering a skin cleansing product that does not leave a slimy, heavy/unfresh feeling. The panel also observed that using Formula 1B resulted in less sensation of skin roughness as compared to Formula 1A (95% confidence interval). This decrease in skin roughness perception means that a skin cleansing product can be rendered that does not leave a feeling of dehydration or skin tightness following its use.

The formulations of Table 15 were generally easier to rinse overall, since they contained a common soap ingredient, potassium laurate. Thus, these formulations were only evaluated for skin-feel after rinsing. The sensory panel observed that feelings of skin roughness immediately after rinsing of Formula 2B were statistically lower than those experienced after rinsing Formula 2A (90% confidence interval). This decrease in skin roughness perception means that a soap product can be rendered that does not leave a feeling of dehydration or skin tightness following its use.

What is claimed is:

1. A composition comprising:
(i) about 15% to 75% by weight of at least one organic solvent,
(ii) about 20% to 50% by weight of at least one cationic alpha-glucan ether derivative, and
(iii) less than about 50% by weight water,
wherein
at least about 50% of the glycosidic linkages of the cationic alpha-glucan ether derivative are alpha-1,6 linkages,
and the cationic alpha-glucan ether derivative has a degree of substitution (DoS) of about 0.001 to about 3.0 with at least one positively charged organic group that is ether-linked to the alpha-glucan.

2. The composition of claim 1, wherein at least about 90% of the glycosidic linkages of the cationic alpha-glucan ether derivative are alpha-1,6 linkages.

3. The composition of claim 1, wherein the cationic alpha-glucan ether derivative comprises at least about 1% alpha-1,2 and/or alpha-1,3 branches.

4. The composition of claim 1, wherein:
(a) the alpha-glucan of said cationic ether derivative has a weight-average molecular weight (Mw) of about 0.9 kDa to 450 kDa, or
(b) the cationic alpha-glucan ether derivative has an Mw of about 1 kDa to 500 kDa.

5. The composition of claim 1, wherein said DoS is about 0.01 to 1.5.

6. The composition of claim 1, wherein the positively charged organic group comprises a substituted ammonium group.

7. The composition of claim 6, wherein the substituted ammonium group comprises a quaternary ammonium group.

8. The composition of claim 7, wherein the quaternary ammonium group comprises a trimethylammonium group.

9. The composition of claim 7, wherein the quaternary ammonium group comprises at least one C₁₀ to C₁₆ alkyl group.

10. The composition of claim 1, wherein the positively charged organic group comprises a quaternary ammonium hydroxyalkyl group.

11. The composition of claim 10, wherein the quaternary ammonium hydroxyalkyl group comprises a trimethylammonium hydroxyalkyl group.

12. The composition of claim 1, wherein said at least one organic solvent comprises ethanol, ethylene glycol, polyethylene glycol, 1,2-propanediol, propylene glycol, dipropylene glycol, tripropyleneglycol, polypropylene glycol, and/or glycerol.

13. The composition of claim 1, comprising:
(i) about 25% to 40% by weight of said at least one organic solvent,
(ii) about 25% to 40% by weight of said at least one cationic alpha-glucan ether derivative, and
(iii) about 25% to 40% by weight water.

14. A product comprising the composition of claim 1, typically wherein the composition was used as an ingredient/component in producing the product.

15. The product of claim 14, wherein the product is a household care product, personal care product, industrial product, or pharmaceutical product.

16. The product of claim 14, wherein the product is (i) a hair shampoo or hair conditioner, or (ii) a skin cleanser, soap, or other skin-washing product.

17. A method of producing a composition according to claim 1, said method comprising:
(a) providing an aqueous composition comprising the cationic alpha-glucan ether derivative,
(b) mixing, into the aqueous composition, the organic solvent, and
(c) optionally concentrating the cationic alpha-glucan ether derivative and the organic solvent in the aqueous composition following step (b).

18. The method of claim 17, wherein the aqueous composition provided in step (a) is an etherification reaction composition in which the cationic alpha-glucan ether derivative was produced.

19. The method of claim 18, wherein step (a) comprises subjecting the etherification reaction composition to one or more rounds of a purification process.
